(19) United States
(12) Patent Application Publication
(10) Pub. No.: US 2026/0224621 A1
(43) Pub. Date: Aug. 6, 2026

(54) METHODS FOR CD48 TARGETED IMMUNOTHERAPY

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Ofer MANDELBOIM, Jerusalem (IL); Rebecca KOTZUR, Jerusalem (IL); Shlomo ELIAS, Jerusalem (IL); Hanan JABER, Jerusalem (IL); Anas ABU KHALAF, Jerusalem (IL)

(21) Appl. No.: 19/152,073

(22) PCT Filed: Jan. 30, 2024
(86) PCT No.: PCT/IL2024/050118

§ 371 Date: Jul. 30, 2025

(30) Foreign Application Priority Data
Jan. 30, 2023 (IL) 300306

(51) Int. Cl. A61K 35/17; A61K 40/15; A61K 40/31; A61K 40/42; A61P 35/00; C07K 14/705; C07K 16/28; C12N 5/0783; C12N 13/00; C12N 15/86

(52) U.S. Cl. CPC A61K 35/17; A61K 40/15; A61K 40/31; A61K 40/4211; A61P 35/00; C07K 14/70535; C07K 14/70596; C07K 16/2803; C12N 5/0646; C12N 13/00; C12N 15/86; A61K 2239/13; A61K 2239/21; A61K 2239/22; C07K 2319/30; C12N 2510/00; C12N 2740/15043

(57) ABSTRACT

Pharmaceutical compositions comprising a non-proliferating cell of an immortalized natural killer (NK) cell line exogenously expressing the lncRNA NeST, CD16 or a functional fragment thereof or an anti-CD19 CAR are provided. The use of the compositions in treating a CD48 expressing cancer in a subject in need thereof, are also provided. Methods for producing a non-proliferating NK cell and pharmaceutical compositions comprising the non-proliferating NK cell obtainable by the methods disclosed herein are also provided.

### REFERENCE TO AN ELECTRONIC SEQUENCE LISTING

The contents of the electronic sequence listing (HUJI-P-095-PCT.xml; Size: 32,912 bytes; and Date of Creation: Jan. 29, 2024) is herein incorporated by reference in its entirety.

### CROSS REFERENCE TO RELATED APPLICATIONS

This application claims the benefit of priority of Israeli Patent Application No. 300306 filed on Jan. 30, 2023, the contents of which are all incorporated herein by reference in their entirety.

### FIELD OF INVENTION

The present invention is in the field of immune cell therapeutics.

### BACKGROUND OF THE INVENTION

NK cells play a pivotal role in anti-cancer immunity, highlighting them as potential therapeutic anticancer agents. Indeed, following the successes of chimeric antigen receptor (CAR)-engineered adoptive T cell therapy, there has been a large interest in NK cells, or CAR-NK cells, as potential candidates for immunotherapy. Several clinical studies utilizing NK cell-based immunotherapies, such as the NK cell line NK-92, are ongoing. Nonetheless, understanding mechanisms underlying NK cytotoxicity, could be utilized to transform NK cells into a more powerful weapon against cancer cells.

The NK cell line, YTS, is derived from NK cell lymphoblastic leukemia. YTS cells express the active receptor 2B4, which is a receptor for CD48. CD48, also known as B-lymphocyte activation marker (BLAST-1) or signaling lymphocytic activation molecule 2 (SLAMF2), is a cell surface receptor member of the CD2 subfamily of the immunoglobulin superfamily (IgSF). CD48 has been reported to be overexpressed in several forms of cancer, including leukemia, lymphoma, multiple myeloma, glioma, breast cancer, and non-small-cell lung cancer. YTS cells also have the added benefit that unlike NK-92 cells they do not require supplementation with IL-2 to grow in culture and thus can be grown more robustly in culture before therapeutic use.

Long non-coding RNAs (lncRNA) are a type of RNA molecule that are not translated into protein. Growing evidence suggests that these molecules possess functional information and regulate many cellular processes. A particular subtype of lncRNAs is the enhancer-like lncRNA, which have a role in the activation of mRNA transcription. One important enhancer-like lncRNA is IFNG-AS1, also known as TMEVPG1 or NeST, that was originally found as a candidate molecule that can explain the differences in murine response to Theiler's virus. Further research showed that NeST regulates IFN-γ expression and secretion. NeST has been largely studied in CD4+ T cells, and specifically, in the Th1 lineage.

It was recently demonstrated that NK cells express a different sequence of NeST as compared to the previously described sequence expressed by T cells. This suggests that a different mechanism for regulation of IFN-γ expression and/or secretion may be active in T cells and NK cells (Stein N et al., “IFNG-AS1 Enhances Interferon Gamma Production in Human Natural Killer Cells”, iScience. 2019; 11:466-473, herein incorporated by reference in its entirety). Moreover, it is known that NK-92 cells retain high cytotoxic activity immediately after irradiation with 10000 cGy, however the cells surviving irradiation lose more than 50% activity, as early as one day after irradiation (Navarrete-Galvan, et al., “Optimizing NK-92 serial killers; gamma irradiation, CD95-Fas-ligation, and NK or LAK attack limit cytotoxicity efficacy”, J. Transl. Med., 2022, 20, 151, herein incorporated by reference in its entirety).

There is a great need for new NK cell-based anticancer immunotherapies. Moreover, taking into consideration the immortalized phenotype of cell lines, it is necessary to find ways to reduce the carcinogenic potential of these immortalized cells, concomitantly with preserving their cytotoxic capability, enabling use of these cells as an efficient and safe anticancer therapy.

### SUMMARY OF THE INVENTION

The present invention provides pharmaceutical compositions comprising a non-proliferating cell of an immortalized natural killer (NK) cell line exogenously expressing the lncRNA NeST, CD16 or a functional fragment thereof or an anti-CD19 CAR. The use of the compositions of the invention in treating a CD48 expressing cancer in a subject in need thereof. Methods for producing a non-proliferating NK cell and pharmaceutical compositions comprising the non-proliferating NK cell obtainable by the methods disclosed herein are also provided.

According to a first aspect, there is provided a pharmaceutical composition comprising a non-proliferating cell of an immortalized natural killer (NK) cell line exogenously expressing the lncRNA NeST.

According to another aspect, there is provided a pharmaceutical composition comprising a non-proliferating cell of an immortalized natural killer (NK) cell line exogenously expressing the lncRNA NeST, for use in treating a CD48 expressing cancer in a subject in need thereof.

According to another aspect, there is provided a pharmaceutical composition comprising a cell of the YTS immortalized NK cell line exogenously expressing CD16 or a functional fragment thereof capable of binding an Fc.

According to another aspect, there is provided a pharmaceutical composition comprising a cell of the YTS immortalized NK cell line exogenously expressing an anti-CD19 chimeric antigen receptor (CAR).

According to some embodiments, the cell comprises a transmembrane receptor for CD48.

According to some embodiments, the receptor is 2B4.

According to some embodiments, the NK cell line is YTS.

According to some embodiments, the NeST comprises the nucleotide sequence as set forth in SEQ ID NO: 1.

According to some embodiments, the cell comprises a lentiviral expression vector encoding the NeST.

According to some embodiments, the CD16 is CD16a and comprises the amino acid sequence of SEQ ID NO: 2.

According to some embodiments, the CD16 is CD16a comprising SEQ ID NO: 2 with a F157V mutation, an S197P mutation or both.

According to some embodiments, the cell comprises a chimeric protein comprising an extracellular domain of CD16, a transmembrane domain and an intracellular immune cell signaling domain.

According to some embodiments, the cell is a non-proliferating cell.

According to some embodiments, the non-proliferating cell is an irradiated cell.

According to some embodiments, the non-proliferating cell is obtainable by a method comprising:
(a) providing a cell of an immortalized NK cell line exogenously expressing a long non-coding RNA (lncRNA) NeST; and,
(b) irradiating the cell of step (a) with ionizing radiation;
thereby, producing a non-proliferating cell.

According to some embodiments, the non-proliferating cell is obtainable by a method comprising:
(a) providing a cell of the YTS immortalized NK cell line exogenously expressing CD16 or a functional fragment thereof capable of binding an Fc; and,
(b) irradiating the cell of step (a) with ionizing radiation;
thereby, producing a non-proliferating cell.

According to some embodiments, the non-proliferating cell is obtainable by a method comprising:
(a) providing a cell of the YTS immortalized NK cell line exogenously expressing an anti-CD19 CAR; and
(b) irradiating the cell of step (a) with ionizing radiation;
   thereby, producing a non-proliferating cell.

According to some embodiments, the irradiating is with a dose sufficient to render the cell of the immortalized NK cell line non-proliferating.

According to some embodiments, the irradiating is with an ionizing radiation dose between 1500 cGY to 6000 cGY.

According to some embodiments, the irradiated is with an ionizing radiation dose between 1500 cGY to 3500 cGY.

According to some embodiments, the pharmaceutical composition is for use in treating a CD48 expressing cancer in a subject in need thereof.

According to some embodiments, the CD48 expressing cancer is selected from: hematopoietic cancer, hepatocellular carcinoma, glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, and melanoma.

According to some embodiments, the CD48 expressing cancer is a hematopoietic cancer.

According to some embodiments, the hematopoietic cancer is a lymphoma.

According to some embodiments, the pharmaceutical composition is for use in treating a CD19 expressing cancer in a subject in need thereof.

According to some embodiments, the pharmaceutical composition is for use in treating an autoimmune disease.

According to some embodiments, the autoimmune disease is selected from systemic sclerosis, lupus, dermatomyositis, multiple sclerosis (MS) and rheumatoid arthritis (RA).

According to some embodiments, the pharmaceutical composition comprises a therapeutically effective amount of non-proliferating cells.

According to some embodiments, the subject in need thereof comprises cancer cells expressing CD48 above a predetermined threshold.

According to some embodiments, the cancer cells expressing CD48 above a predetermined threshold are in a sample of the subject in need thereof, selected from: peripheral blood, plasma, serum, tumor biopsy, tumor fluid or any combination thereof.

According to another aspect, there is provided a method of treating a CD48 expressing cancer in a subject in need thereof, the method comprising administering to the subject a pharmaceutical composition of the invention, thereby treating a CD48 expressing cancer.

According to another aspect, there is provided a method of treating a CD19 expressing cancer in a subject in need thereof, the method comprising administering to the subject a pharmaceutical composition of the invention, thereby treating a CD19 expressing cancer.

According to another aspect, there is provided a method of treating an autoimmune disease in a subject in need thereof, the method comprising administering to the subject a pharmaceutical composition of the invention, thereby treating an autoimmune disease.

According to another aspect, there is provided a method for producing a non-proliferating natural killer (NK) cell, the method comprising:
(a) providing a cell of an immortalized NK cell line exogenously expressing a long non-coding RNA (lncRNA) NeST; and,
(b) irradiating the cell of step (a) with ionizing radiation;
thereby, producing a non-proliferating NK cell.

According to another aspect, there is provided a method for producing a non-proliferating YTS cell, the method comprising:
(a) providing a cell of the immortalized NK cell line YTS exogenously expressing CD16 or a functional fragment thereof capable of binding an Fc or an anti-CD19 CAR; and,
(b) irradiating the cell of step (a) with ionizing radiation;
thereby, producing a non-proliferating YTS cell.

According to some embodiments, the irradiating is with a dose sufficient to render the cell of the immortalized NK cell line non-proliferative.

According to some embodiments, the irradiating is with an ionizing radiation dose between 1500 cGY to 6000 cGY.

According to some embodiments, the irradiating is with an ionizing radiation dose between 1500 cGY to 3500 cGY.

According to some embodiments, the cell comprises a transmembrane receptor for CD48.

According to some embodiments, the transmembrane receptor is 2B4.

According to some embodiments, the NK cell line is YTS.

According to some embodiments, the NeST comprises the nucleotide sequence as set forth in SEQ ID NO: 1.

According to some embodiments, the cell comprises a lentiviral expression vector encoding the NeST.

According to some embodiments, the CD16 is CD16a and comprises the amino acid sequence of SEQ ID NO: 2.

According to some embodiments, the CD16 is CD16a comprising SEQ ID NO: 2 with a F157V mutation, an S197P mutation or both.

According to some embodiments, the cell comprises a chimeric protein comprising an extracellular domain of CD16, a transmembrane domain and an intracellular immune cell signaling domain.

According to some embodiments, the non-proliferating is non-cancerous.

According to some embodiments, the non-proliferating NK cell is further characterized by: increased cytotoxic activity against a target cell, increased inflammatory activity, or both, as compared to a control cell of the immortalized NK cell not exogenously expressing NeST.

According to some embodiments, the non-proliferating NK cell is further characterized by: increased cytotoxic activity against a target cell, increased inflammatory activity, or both, as compared to a control YTS cell not expressing CD16 or a functional fragment thereof capable of binding an Fc.

According to some embodiments, the non-proliferating NK cell is further characterized by: increased cytotoxic activity against a target cell, increased inflammatory activity, or both, as compared to a control YTS cell not expressing an anti-CD19 CAR.

According to some embodiments, the target cell is a cancer cell expressing CD48.

According to some embodiments, the target cell is a cancer cell expressing CD19.

According to some embodiments, the method further comprises after the irradiating measuring proliferation of the cell and selecting a cell that does not proliferate.

According to another aspect, there is provided a pharmaceutical composition comprising a non-proliferating NK cell obtainable by a method of the invention.

According to another aspect, there is provided a pharmaceutical composition comprising a non-proliferating NK cell produced by a method of the invention.

According to another aspect, there is provided a pharmaceutical composition comprising a non-proliferating YTS cell obtainable by a method of the invention.

According to another aspect, there is provided a pharmaceutical composition comprising a non-proliferating YTS cell produced by a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Expression of NeST in YTS cells. YTS cells were transduced with NeST (YTS OE NeST). Expression of NeST was verified using qRT-PCR. The relative quantity (RQ) was based on a GAPDH control gene expression and normalized to an independent sample of untreated parental YTS cells (YTS par).

FIGS. 2A-2B: Expression of CD48 on the surface of target cells. (2A) Cell lines were stained with anti-human CD48-APC antibody. Black line histograms indicate CD48 expression, whereas the gray histograms indicate APC-background control. The cell lines 721.221, RAJI, 8866, BCBL-1, BJAB, and Jurkat were found to express CD48. (2B) Human protein atlas bar graph showing cancer cells expressing CD48. CD48 is present only in lymphoma cells, myeloma cells and to a lesser extent leukemia cells.

FIGS. 3A-3B: Comparative secretion of IFN-γ by YTS cells overexpressing NeST and parental YTS cells, in response to activation with CD48 expressing cell lines and CD48 negative cell line. YTS cells overexpressing NeST (YTS OE NeST), or parental YTS cells (YTS par) were incubated (3A) with target cells expressing CD48, BCBL1 and 721.221, or (3B) with CD48 negative cell line K562 for 48 h in an E:T ratio of 0.5:1. Supernatants were collected for detection of IFN-γ by ELISA. Activation of YTS OE NeST, and not YTS par cells, with the CD48 expressing cell lines; 721.221 or BCBL 1, significantly induced IFN-γ secretion, whereas incubation of the YTS OE NeST cells with K562 cells did not lead to any significant change in IFN-γ production. Student's T-test, two-tailed; *=p<0.05; **=p<0.01.

FIG. 4: Comparative killing capacities of YTS cells overexpressing NeST and parental YTS cells. YTS cells and radioactively labeled target cells BJAB, 721.221 and K562 were incubated for 5 h using E:T ratios as presented in the X axis. There was a significant increase in YTS OE NeST killing capacity, compared to the killing capacity of YTS par, of both CD48 expressing cell lines: 721.221 and BJAB, in both presented E:T ratios. No induction of killing capacity was demonstrated for the YTS OE NeST cells when incubated with the CD48 negative cell line K562. Student's T-test, two-tailed, *=p<0.05.

FIG. 5: MTT proliferation assay of irradiated YTS cells. YTS OE NeST cells were irradiated with 1,000, 2,000, 3,000, and 6,000cGy and their viability was examined by MTT assay. No induced proliferation was observed for YTS cells irradiated with 2000cGy, 3000cGy and 6000cGy.

FIGS. 6A-6D: Comparative secretion of IFN-γ by 2000 and 3000 cGy-irradiated YTS cells overexpressing NeST and irradiated parental YTS cells, in response to activation with CD48 expressing cell lines and CD48 negative cell line. (6A-6C) YTS cells, either overexpressing NeST, infected with an empty vector (EV) or parental YTS cells, were irradiated with an irradiation dose of (6A-6B) 2,000 cGy or (6C) 3000 cGy before incubation with the CD48 positive target cells (6A, 6C) BCBL-1 and (6B, 6C) 721.221, for 48 hours at an E:T ratio of 0.5:1 or 1:1. Supernatants were collected for examination of IFN-γ level by ELISA. (6D) YTS parental cells and NeST overexpressing cells irradiated with 2,000 cGy were also incubated with the CD48 negative target cell line K562 at an E:T ratio of 0.5:1 and after 48 hours IFN-γ in supernatants was measured. Statistical analysis: Student's T-test, two-tailed; *=p<0.05; **=p<0.01; NS-not significant.

FIGS. 7A-7B: Comparative killing capacities of 3000cGy-irradiated YTS cells overexpressing NeST and the corresponding irradiated parental YTS cells. 3000 cGy irradiated YTS cells and radioactively labeled target cells BJAB, 721.221 and K562 were incubated for 5 h using E:T ratios as presented in the X axis. (7A) There was a significant increase in the irradiated YTS NeST killing capacity, compared to the killing capacity of the irradiated YTS par, for both CD48 expressing cell lines: 721.221 and BJAB, in all presented E:T ratios. (7B) No increase in killing capacity was demonstrated for the irradiated YTS OE NeST cells when incubated with the CD48 negative cell line K562. Student's T-test, two-tailed, *=p<0.05; NS: non-significant.

FIG. 8: Weight analysis of mice administered with irradiated and non-irradiated NK cells. Bar graph showing the average weight of mice after injection of YTS parental cells (non-irradiated, 2000 cGY, and 3000 cGY), seYTS cells (non-irradiated, 2000 cGY, and 3000 cGY) and NK-92 cells (non-irradiated). Mice treated with non-irradiated YTS parental cells and seYTS cells were all dead by day 25.

FIG. 9: Bar graph of average tumor weight in mice injected with non-irradiated YTS parental cells or YTS NeST cells.

FIG. 10: In-vivo effect of intravenous administration of irradiated YTS NeST (seYTS) in a SCID beige mice tumor model. 10×10\hat{ }6 721.221 tumor cells were subcutaneously injected to SCID beige mice. At day 27 post tumor cell injection, mice were intravenously injected with 2×10\hat{ }6 of either irradiated YTS parental cells (YTSpar), irradiated YTS NeST cells (seYTS), or irradiated NK-92 cells, in 100 μl PBS. Irradiation was at a dose of 2000 cGY. PBS alone treated mice served as negative controls. Statistical analysis: Student's T-test, two-tailed; *=p<0.05; **=p<0.01. Statistical significance is provided as significance is with respect to 721 221 alone/significance is with respect to 721 221 YTSpar.

FIGS. 11A-11D: Comparative killing capacities of YTS cells overexpressing CD16 and control YTS cells. YTS cells and radioactively labeled (11A-11B) Raji cells or (11C-11D) primary CLL cells were incubated first with either Herceptin, Rituximab or no antibody. The cells were then incubated with (11A, 11C) control YTS cells expressing an empty vector or with (11B, 11D) YTS-CD16 cells and specific killing of the target cells was measured. Specific cell killing of the CD48 and CD20 positive cells was observed only for the YTS-CD16 cultures.

FIGS. 12A-12B: Comparative killing capacities of irradiated and non-irradiated YTS cells overexpressing CD16. Radioactively labeled target Raji cells pre-contacted with Herceptin, Rituximab or no antibody and then were incubated with (12A) non-irradiated YTS-CD16 cells or (12B) irradiated YTS-CD16 cells and specific killing of the target cells was measured.

FIGS. 13A-13C: Comparative killing capacities of YTS cells overexpressing CAR-CD19, control YTS cells and irradiated versions thereof. (13A-13B) YTS cells and radioactively labeled (13A) Raji cells or (13B) primary CLL cells were incubated with control YTS cells expressing an empty vector or with YTS-CAR-CD19 cells and specific killing of the target cells was measured. (13C) Irradiated and non-irradiated WT YTS cells (WT), YTS cells expressing CAR-CD19 (CARCD19) and YTS cells expressing an empty vector (EV) were cultured with radioactively labeled Raji cells and specific target cell killing was measured.

FIGS. 14A-14B: Comparative killing capacities of YTS cells overexpressing CAR-CD19 after various doses of radiation. Radioactively labeled target Raji cells incubated with YTS-CAR-CD19 cells irradiated with various levels of radiation and specific killing was measured (14A) on the same day as the coculture and (14B) 24 hours after initiation of the coculture.

FIG. 15: Comparative killing capacities of melanoma cells by YTS cells expressing and not expressing CAR-CD19. Bar graph of specific killing of melanoma cell lines A357, MEL 526 and MEL 624 with and without exogenous overexpression of CD19 by YTS cells expressing an empty vector (EV) or an anti-CD19 CAR (CAR).

### DETAILED DESCRIPTION OF THE INVENTION

The present invention, in some embodiments, provides a pharmaceutical composition comprising a non-proliferating cell of an immortalized natural killer (NK) cell line exogenously expressing the lncRNA NeST, for use in treating a CD48 expressing cancer in a subject in need thereof. Methods for producing a non-proliferating NK cell, are also provided.

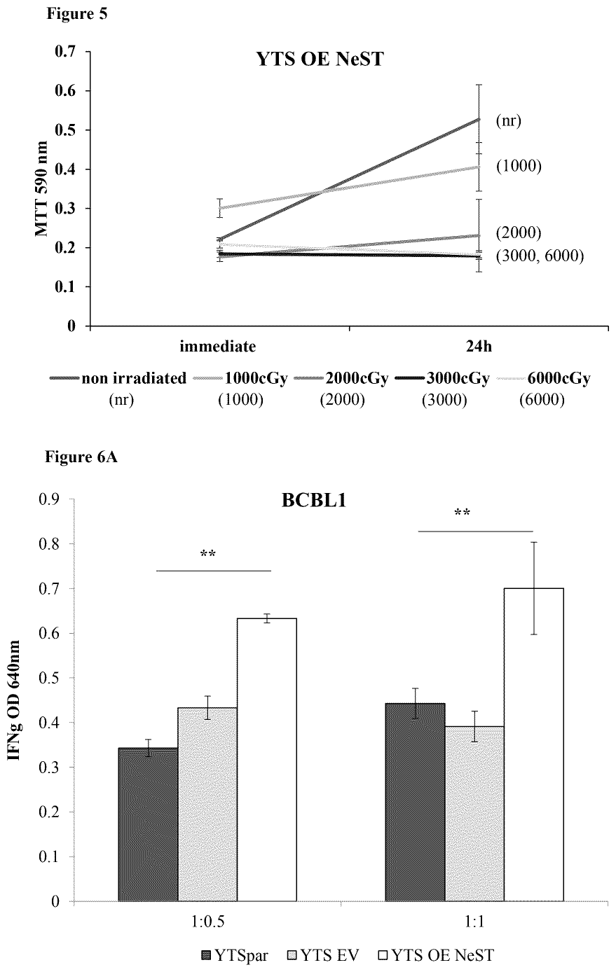

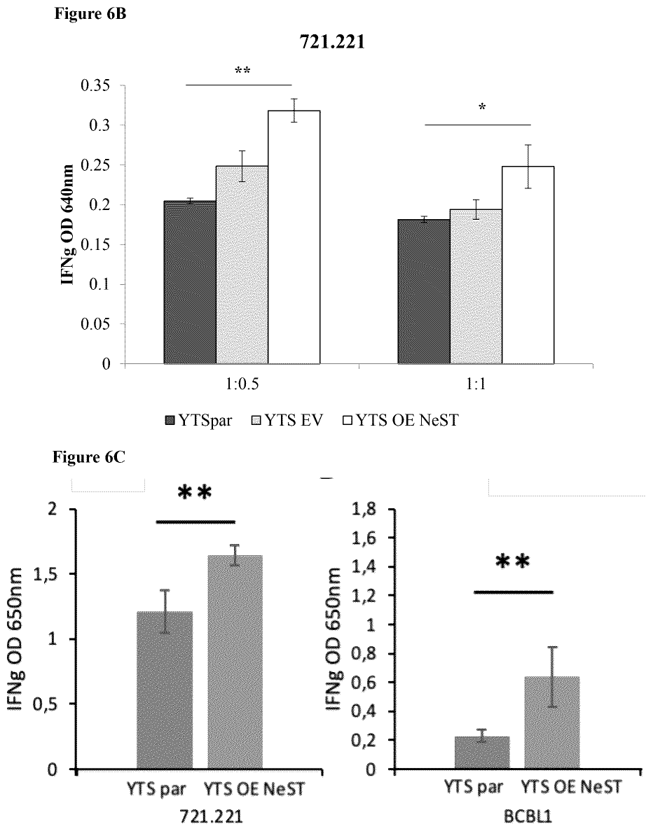

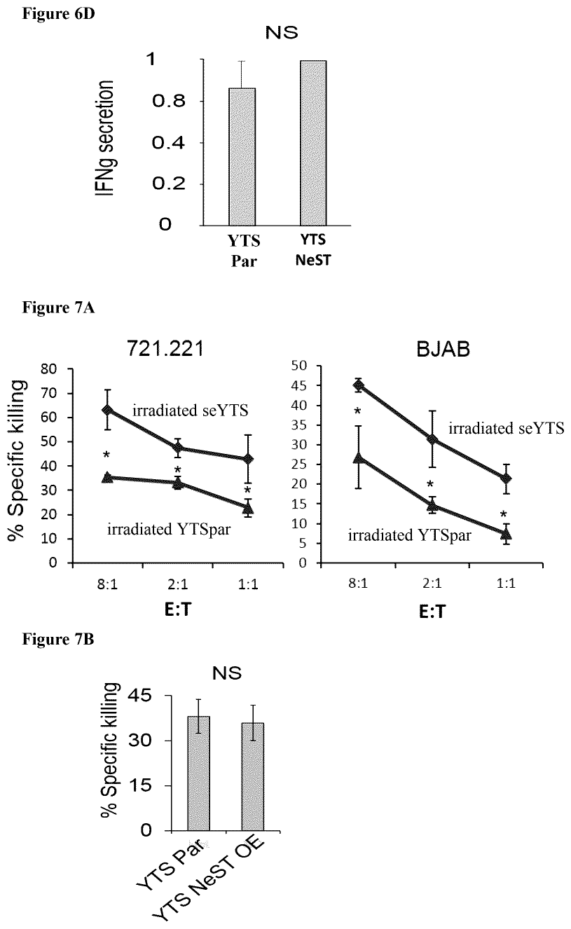

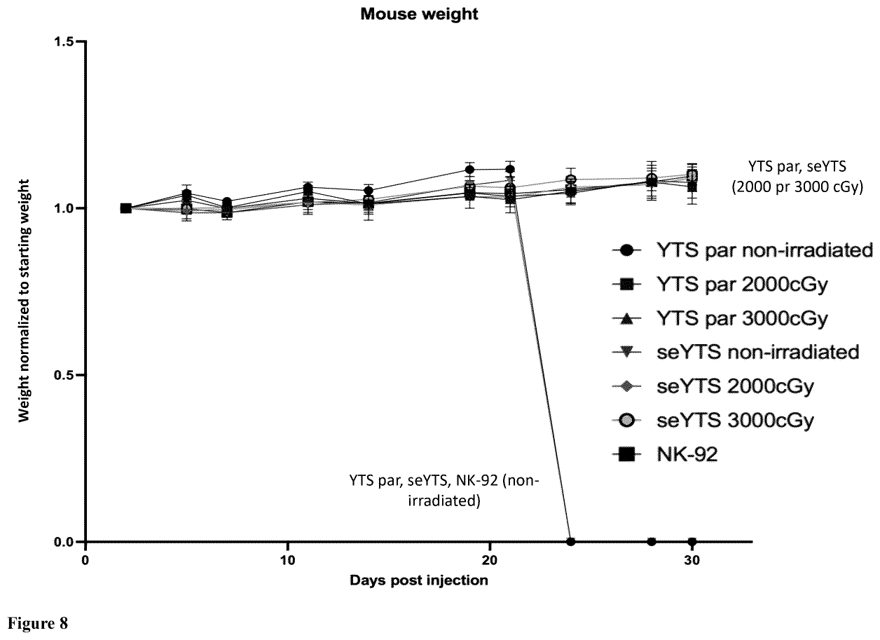

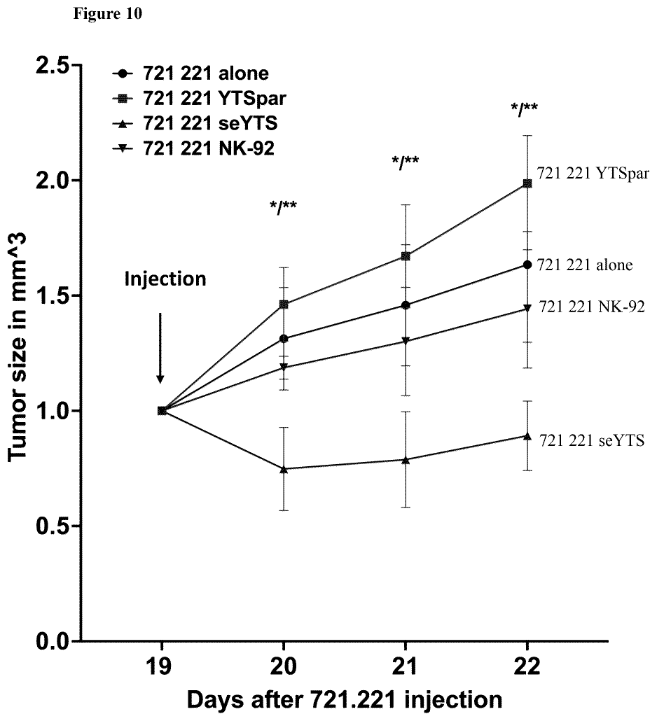

The invention is based, at least in part, on the surprising finding that an immortalized cell of an NK cell line, such as YTS, exogenously expressing the long non-coding RNA (lncRNA) NeST (seYTS), successfully retains its cytotoxic activity, manifested by increased IFN-γ secretion and killing capacity of CD48 expressing cancer cell (e.g., lymphoma, when irradiated with an ionizing radiation dose of 2000 cGY (FIGS. 6A-6B, and 7A-7B). This finding is unexpected taking into consideration that other NK cell lines, such as NK-92, expressing endogenous levels of NeST, lose as much as 50% of their cytotoxic activity, when irradiated with a radiation dose of 1000 cGY. Further and in contrast to wild-type NK-92, a radiation dose of 1000 cGY was not effective in rendering the seYTS cells non-proliferative (FIG. 5). The invention is further based on the surprising finding that in vivo treatment of mice with the irradiated seYTS cells significantly reduced tumor size compared to both vehicle- and NK-92 cell treated groups (FIG. 10), concomitantly with no carcinogenic or other adverse effects (FIG. 8).

### Compositions and Use Thereof

By a first aspect, there is provided a pharmaceutical composition comprising a non-proliferating cell of an immortalized natural killer (NK) cell line exogenously expressing the long non-coding RNA (lncRNA) NeST.

By another aspect, there is provided a pharmaceutical composition comprising a cell of an immortalized NK cell line exogenously expressing CD16 or an Fc-binding fragment thereof.

By another aspect, there is provided a pharmaceutical composition comprising a cell of an immortalized NK cell line exogenously expressing an anti-CD19 chimeric antigen receptor (CAR).

By another aspect, there is provided a method of treating cancer in a subject in need thereof, the method comprising administering to the subject a pharmaceutical composition of the invention, thereby treating cancer in a subject.

By another aspect, there is provided a method of treating an autoimmune disease in a subject in need thereof, the method comprising administering to the subject a pharmaceutical composition of the invention, thereby treating an autoimmune disease in a subject.

In some embodiments, the pharmaceutical composition is for use in treating a cancer in a subject in need thereof. In some embodiments, the pharmaceutical composition if for use in the production of a medicament for treating cancer. In some embodiments, the cancer is in a subject. In some embodiments, the subject suffers from cancer. In some embodiments, the cancer is a CD48 expressing cancer. In some embodiments, the cancer is a CD19 expressing cancer.

In some embodiments, the pharmaceutical composition is for use in treating an autoimmune disease in a subject in need thereof. In some embodiments, the pharmaceutical composition if for use in the production of a medicament for treating an autoimmune disease.

In some embodiments, the autoimmune disease is in a subject. In some embodiments, the subject suffers from an autoimmune disease. In some embodiments, the autoimmune disease is an autoimmune disease treatable by CAR therapy. In some embodiments, CAR therapy is CAR-T therapy. In some embodiments, CAR therapy is CAR-NK therapy. In some embodiments, the composition comprising CAR-CD19 is used to treat an autoimmune disease. In some embodiments, the composition comprising CD16 is used to treat an autoimmune disease. In some embodiments, the composition comprising NeST is used to treat an autoimmune disease. In some embodiments, the autoimmune disease is selected from systemic sclerosis (scleroderma), lupus, dermatomyositis, multiple sclerosis (MS) and rheumatoid arthritis (RA). In some embodiments, lupus is systemic lupus erythematosus (SLE). In some embodiments, the composition comprising CAR-CD19 is used in treating an autoimmune disease selected from scleroderma, lupus, dermatomyositis and MS. In some embodiments, the composition comprising CD16 is used to treat an autoimmune disease selected from MS and RA. In some embodiments, the autoimmune disease is scleroderma.

In some embodiments, the autoimmune disease is lupus. In some embodiments, the autoimmune disease is dermatomyositis. In some embodiments, the autoimmune disease is MS. In some embodiments, the autoimmune disease is RA.

As used herein, the term “an immortalized NK cell line” is well known in the art and encompasses a population of NK cells which potentially can proliferate indefinitely. In some embodiments, the immortalized NK cell line comprises a mutation that is responsible for its immortality. In some embodiments, the immortalized NK cell line originated from a malignant cancer. In some embodiments, the cancer is a solid cancer. In some embodiments, the cancer is a tumor. In some embodiments, the malignant cancer is leukemia. In some embodiments, the leukemia is an NK leukemia. In some embodiments, the malignant cancer is lymphoma. In some embodiments, the lymphoma is an NK lymphoma. In some embodiments, the immortalized NK cell line is generated in vitro. In some embodiments, the immortalized NK cell line is from a blood sample. In some embodiments, the blood sample is from a healthy donor. In some embodiments, the immortalized cell line is from pericardial fluid. In some embodiments, the immortalized NK cell line is mammalian. In some embodiments, the immortalized NK cell line is from a human subject. In some embodiments, the immortalized NK cell line is derived from a NK cell lymphoblastic leukemia/lymphoma. In some embodiments, the immortalized NK cell line is selected from: YTS (accession #CVCL_D324), NK92 (accession #CVCL_2142), NK3.3 (accession #CVCL_7994), NKL (accession #CVCL_0466), SRIK-NKL (accession #CVCL_IP68), or any combination thereof. In some embodiments, YTS is YTS-Eco (accession #CVCL_EG36).

In some embodiments, the immortalized NK cell line comprises a YT cell line (accession #CVCL_1797). The YT cell line is a known cell line derived from human lymphoblastic leukemia/lymphoma. In some embodiments, the immortalized NK cell line comprises a subtype of the YT cell line. In some embodiments, the immortalized NK cell line is a subtype of the YT cell line. In some embodiments, the immortalized NK cell line comprises a YTS cell line. In some embodiments, the immortalized NK cell line is a YTS cell line. The YTS cell line (accession #CVCL_D324) is a known cell line derived from human lymphoblastic leukemia/lymphoma. In some embodiments, the immortalized NK cell line is not NK-92.

In some embodiments, the immortalized NK cell line is non-proliferating. In some embodiments, the cell is not proliferating. In some embodiments, the cell is a non-proliferative cell. As used herein, the term “non-proliferating”, “non-proliferative”, and “non-dividing” are used interchangeably. In some embodiments, a non-proliferating cell is a cell that does not divide. In some embodiments, a non-proliferating cell is a cell that does not proliferate for at least 16 hr. In some embodiments, a non-proliferating cell is a cell that does not proliferate for a time period between 16 hr to 144 hr, 16 hr to 132 hr, 16 hr to 120 hr, 16 hr to 108 hr, 16 hr to 96 hr, between 16 hr to 84 hr, between 16 hr to 72 hr, between 16 hr to 60 hr, between 16 hr to 48 hr, between 16 hr to 32 hr, or between 16 hr to 24 hr.

Each possibility represents a separate embodiment of the present invention. In some embodiments, a non-proliferating cell comprises a cell in a quiescent condition. As used herein, the term “quiescence condition” refers to a cellular state in which a cell remains out of the cell cycle. In some embodiments, the cell retains the capacity to divide. In some embodiments, the cell does not retain the capacity to divide. In some embodiments, a non-proliferating cell is a cell that does not proliferate when it is not activated. In some embodiments, a non-proliferating cell is a cell that does not proliferate terminally.

In some embodiments, a non-proliferating cell is a non-cancerous cell. As used herein, the term “non-cancerous cell” encompasses a cell that does not induce cancer or tumor growth when administered to a subject in need thereof. In some embodiments, a non-cancerous cell is a cell that does not transform into a cancer cell in the subject. In some embodiments, a non-cancerous cell is a cell that does not induce a metastatic tumor in the subject.

As used herein, the term “long non-coding RNA (lncRNA)” encompasses an RNA molecule that is not translated into a protein and is over 50 nucleotides in length. In some embodiments, the lncRNA molecule comprises a nucleotide sequence of more than 200 nucleotides. Examples for lncRNA molecules are known in the art and include intergenic IncRNAs, intronic ncRNAs, sense lncRNA, antisense lncRNA, bidirectional lncRNA, and enhancer-like lncRNA. In some embodiments, the lncRNA is an enhancer-like lncRNA. As used herein, “an enhancer-like IncRNA” is a IncRNA molecule that increases RNA transcription, increases protein translation, or both. In some embodiments, the lncRNA molecule is overexpressed. In some embodiments, exogenous expression of the lncRNA molecule increases protein expression, secretion, or both. In some embodiments, increased protein expression and/or secretion is compared to a cell not exogenously expressing the lncRNA molecule. In some embodiments, the lncRNA disclosed herein comprises Nettoie Salmonella pas Theiler's; cleanup Salmonella not Theiler's (NeST). In some embodiments, the lncRNA is NeST. As used herein, the terms “NeST”, “Tmevpg1”, “IFNG antisense RNA 1” and “IfngAS1” are used interchangeably. In some embodiments, the lncRNA disclosed herein comprises mammalian NeST. In some embodiments, the lncRNA comprises a human NeST.

In some embodiments, the polynucleotide encoding the lncRNA molecule comprises the nucleotide sequence of “Homo sapiens IFNG antisense RNA 1 transcript variant 1 antisense RNA (IFNG-AS1/NeST)”, accession #MK296539. In some embodiments, the polynucleotide encoding the NeST comprises the sequence: ctgcaatttcaggtagcttttctgactcttaaagagatctcaagtataccttcagagaaatgccagcaaaaactgtagtcatttgggaa ggaataagcctggaagaaaaagatacaacgaactagcacaacgaggagtttgaaaagttcatgacagctcacagctgatgatggt ggcaatcttaaggatacagaaagctcattcctcatgcagggaagaagaaaatattctaaagaagagataagcatattccatgaaatc aaaaaagcataaaacgctggaggagaagtcagtagcaagcagctaagacaacatggtacatgtggctagaagcccaccaactg ctaacaaccagctggagggtaagtcaaaagctatgagagcccagaaggagaacaattatttagacctggaggggccagttggga aagtctttgaagggtcttcagaaaatggaccaaaagaaaagaggaaaagtcattctactgagaggtgtttgtgtaatcaaagcaacc gaggcctaagaactcacaaaagattagaaaagggtaaaggatgcctggaaagccggactaggaaaaggctgtgaagggccctg aaggtcacatgtcacatgttgcctgcagcatgcagaatcaaaacatgataaatcatgtcttgaatcatgacatatgaagcatgacaca cgaatcatgacatgtgacatgttgaatgtcacatgtcacatgccgtaggcagccatcaagagtatttgaaaggaaagtgtataatcag gcctgtgctttagtaagacaatttgtatagtgttcagtgtggattaaaatggagaacacaggtatcagtttgacatcgacagtacagga aaccatttgggtttcttgtacaccaaaatattaaaacatacacgcaaacacaccaacacattaaaatcttgagagagagggccaaaa gttctttgtcgctggactaaaagtggaaataaatgtttacaacctctaaggtggattaagctaagaatttggagttgttggcaaaactgc cacataaaatttcagacatgcaaagaaatgcaatcgtagacttatgcacatacttccaccagagaagggaatctgggcagagtgttc aaagcttaagaaattcctttgctcttcaccaagaagagccaacagagttttcgttgaatcaactcttatattcccttaaaaagaagttgat agtgttgtggaaaactcacccattgaagtgacaacaaaaacagcaacccagtatgagttaactggtattatgttaaagggacaagta actgcacttttgacacacatcctctaaaaggtagggtgggagtgtttcaaagaaccaagtctgagtaatagaaaagcatataccactt tcccatatacacgttcgagcatgggggagcaggaagctgggtaattgaatgcagttgtgcacactggcaattttcccgccacatacc atttcttcggtgtctctcccaaaattctcctcctaagtggcacatccctttattgatcatctttccccatactgatcatcctcagcaatcatg tgagttattagtctaagtatttgtttttctcaaatatggtcttttaactactccctaaatattttatatatacatagtttgttttcccaatttaacta caagcatttaaaaaatgggaatatttgtctatatttctgtaatttttcatcatgccttatatggtatataaagatcaacagcaataaaacaac aacagtaataatagctaagaccttttgaatactaactgtatgttcagcattatttcaacagctttacaggtattaactgatttaattattaca acaacggcatgaagtagatactactgttgtcatcagtttatacttgaggaaactgagttatgggatggttaattatcttttccatggtcac agagccattaaatgtatgatctcagacagtc (SEQ ID NO: 1). In some embodiments, the molecule is DNA. In some embodiments, the molecule is RNA.

CD16 is also known as Fc fragment of IgG, low affinity III receptor (FcγRIII). In some embodiments, CD16 is CD16a. In some embodiments, CD16 is CD16b. In some embodiments, CD16 is CD16a or CD16b. In some embodiments, CD16 is selected from CD16a and CD16b. CD16a is also known as FCGR3A and CD16b is also known as FCGR3B. In some embodiments, the CD16 is mammalian CD16. In some embodiments, mammalian CD16 is human CD16. In some embodiments, human CD16a is identified by Entrez gene #2214. In some embodiments, human CD16b is identified by Entrez gene #2215. In some embodiments, human CD16a protein is identified by UniProt ID P08637. In some embodiments, human CD16b protein is identified by UniProt ID 075015. In some embodiments, human CD16a comprises or consists of the nucleotide sequence provided in NM_000569. In some embodiments, human CD16b comprises or consists of the nucleotide sequence provided in NM_000570. In some embodiments, human CD16a comprises or consists of an amino acid sequence provided in NP_000560. In some embodiments, human CD16b comprises or consists of an amino acid sequence provided in NP_000561. In some embodiments, the amino acid sequence of human CD16a comprises MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDFYIPKATL KDSGSYFCRGLFGSKNVSSETVNITITQGLAVSTISSFFPPGYQVSFCLVMVLLFAV DTGLYFSVKTNIRSSTRDWKDHKFKWRKDPQDK (SEQ ID NO: 2). In some embodiments, the amino acid sequence of human CD16a consists of SEQ ID NO: 2. In some embodiments, the amino acid sequence of human CD16b comprises MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDFHIPKATL KDSGSYFCRGLVGSKNVSSETVNITITQGLAVSTISSFSPPGYQVSFCLVMVLLFAV DTGLYFSVKTNI (SEQ ID NO: 3). In some embodiments, the amino acid sequence of human CD16b consists of SEQ ID NO: 3.

In some embodiments, the cell exogenously expresses a fragment of CD16. In some embodiments, the fragment is a functional fragment. In some embodiments, the fragment is an Fc-binding fragment. In some embodiments, the Fc is the Fc portion of an antibody. In some embodiments, an Fc is an Fc fragment. In some embodiments, the antibody is an IgG antibody. In some embodiments, the antibody is a human antibody. In some embodiments, the Fc is a human Fc. In some embodiments, binding of an Fc by an active fragment activates antibody-dependent cell-mediated cytotoxicity (ADCC) in the cell.

In some embodiments, the fragment comprises the CD16 extracellular domain (ECD). In some embodiments, the fragment is a fragment of the CD16 ECD. In some embodiments, the CD16a ECD comprises amino acids 17-208 of SEQ ID NO: 2. In some embodiments, the CD16a ECD consists of amino acids 17-208 of SEQ ID NO: 2. In some embodiments, the CD16a ECD comprises the amino acid sequence GMRTEDLPKAVVFLEPQWYRVLEKDSVTLKCQGAYSPEDNSTQWFHNESLISSQA SSYFIDAATVDDSGEYRCQTNLSTLSDPVQLEVHIGWLLLQAPRWVFKEEDPIHLR CHSWKNTALHKVTYLQNGKGRKYFHHNSDFYIPKATLKDSGSYFCRGLFGSKNV SSETVNITITQGLAVSTISSFFPPGYQ (SEQ ID NO: 8). In some embodiments, the CD16a ECD consists of SEQ ID NO: 8. In some embodiments, the CD16a ECD comprises the CD16a signal peptide and comprises amino acids 1-208 of SEQ ID NO: 2. In some embodiments, the CD16a ECD comprises the CD16a signal peptide and consists of amino acids 1-208 of SEQ ID NO: 2. In some embodiments, the CD16a ECD comprises the CD16a signal peptide and comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDFYIPKATL KDSGSYFCRGLFGSKNVSSETVNITITQGLAVSTISSFFPPGYQ (SEQ ID NO: 10). In some embodiments, the CD16a ECD comprises the CD16a signal peptide and consists of SEQ ID NO: 10. In some embodiments, the CD16b ECD comprises amino acids 17-208 of SEQ ID NO: 3. In some embodiments, the CD16b ECD consists of amino acids 17-208 of SEQ ID NO: 3. In some embodiments, the CD16b ECD comprises the amino acid sequence GMRTEDLPKAVVFLEPQWYSVLEKDSVTLKCQGAYSPEDNSTQWFHNESLISSQA SSYFIDAATVNDSGEYRCQTNLSTLSDPVQLEVHIGWLLLQAPRWVFKEEDPIHLR CHSWKNTALHKVTYLQNGKDRKYFHHNSDFHIPKATLKDSGSYFCRGLVGSKNV SSETVNITITQGLAVSTISSFSPPGYQ (SEQ ID NO: 11). In some embodiments, the CD16b ECD consists of SEQ ID NO: 11. In some embodiments, the CD16b ECD comprises the CD16a signal peptide and comprises amino acids 1-208 of SEQ ID NO: 3. In some embodiments, the CD16b ECD comprises the CD16b signal peptide and consists of amino acids 1-208 of SEQ ID NO: 3. In some embodiments, the CD16b ECD comprises the CD16a signal peptide and comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDFHIPKATL KDSGSYFCRGLVGSKNVSSETVNITITQGLAVSTISSFSPPGYQ (SEQ ID NO: 11). In some embodiments, the CD16b ECD comprises the CD16a signal peptide and consists of SEQ ID NO: 11.

In some embodiments, the CD16 comprises at least one mutation. In some embodiments, the CD16 ECD comprises at least one mutation. In some embodiments, the CD16 fragment comprises at least one mutation. In some embodiments, the mutation increases Fc binding. In some embodiments, the mutation increases affinity for the Fc. In some embodiments, the mutation is mutation of phenylalanine 157 (F157). In some embodiments, the mutation is to valine (F157V). In some embodiments, F157 is with respect to SEQ ID NO: 2. In some embodiments, F157 is with respect to SEQ ID NO: 3. In some embodiments, the CD16a comprising the F157V mutation comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDVYIPKATL KDSGSYFCRGLFGSKNVSSETVNITITQGLAVSTISSFFPPGYQVSFCLVMVLLFAV DTGLYFSVKTNIRSSTRDWKDHKFKWRKDPQDK (SEQ ID NO: 12). In some embodiments, the CD16a comprising the F157V mutation consists of SEQ ID NO: 12. In some embodiments, the CD16b comprising the F157V mutation comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDVHIPKATL KDSGSYFCRGLVGSKNVSSETVNITITQGLAVSTISSFSPPGYQVSFCLVMVLLFAV DTGLYFSVKTNI (SEQ ID NO: 13). In some embodiments, the CD16b comprising the F157V mutation consists of SEQ ID NO: 13.

In some embodiments, the mutation renders the CD16 or fragment thereof resistant to cleavage. In some embodiments, the cleavage is enzymatic cleavage. In some embodiments, the cleavage is ADAM-17 cleavage. In some embodiments, the mutation is mutation of serine 197 (5197). In some embodiments, the mutation is to proline (S197P). In some embodiments, S197 is with respect to SEQ ID NO: 2. In some embodiments, S197 is with respect to SEQ ID NO: 3. In some embodiments, the CD16a comprising the S197P mutation comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDFYIPKATL KDSGSYFCRGLFGSKNVSSETVNITITQGLAVPTISSFFPPGYQVSFCLVMVLLFAV DTGLYFSVKTNIRSSTRDWKDHKFKWRKDPQDK (SEQ ID NO: 14). In some embodiments, the CD16a comprising the S197P mutation consists of SEQ ID NO: 14. In some embodiments, the CD16b comprising the S197P mutation comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDFHIPKATL KDSGSYFCRGLVGSKNVSSETVNITITQGLAVPTISSFSPPGYQVSFCLVMVLLFAV DTGLYFSVKTNI (SEQ ID NO: 15). In some embodiments, the CD16b comprising the S197P mutation consists of SEQ ID NO: 15.

In some embodiments, the CD16a comprising the F157V mutation and the S197P mutation comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDVYIPKATL KDSGSYFCRGLFGSKNVSSETVNITITQGLAVPTISSFFPPGYQVSFCLVMVLLFAV DTGLYFSVKTNIRSSTRDWKDHKFKWRKDPQDK (SEQ ID NO: 16). In some embodiments, the CD16a comprising the F157V mutation and the S197P mutation consists of SEQ ID NO: 16. In some embodiments, the CD16b comprising the F157V mutation and the S197P mutation comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDVHIPKATL KDSGSYFCRGLVGSKNVSSETVNITITQGLAVPTISSFSPPGYQVSFCLVMVLLFAV DTGLYFSVKTNI (SEQ ID NO: 17). In some embodiments, the CD16b comprising the F157V mutation and the S197P mutation consists of SEQ ID NO: 17.

In some embodiments, the CD16a ECD comprises amino acids 17-208 of SEQ ID NO: 12. In some embodiments, the CD16a ECD consists of amino acids 17-208 of SEQ ID NO: 12. In some embodiments, the CD16a ECD comprises the amino acid sequence GMRTEDLPKAVVFLEPQWYRVLEKDSVTLKCQGAYSPEDNSTQWFHNESLISSQA SSYFIDAATVDDSGEYRCQTNLSTLSDPVQLEVHIGWLLLQAPRWVFKEEDPIHLR CHSWKNTALHKVTYLQNGKGRKYFHHNSDVYIPKATLKDSGSYFCRGLFGSKNV SSETVNITITQGLAVSTISSFFPPGYQ (SEQ ID NO: 18). In some embodiments, the CD16a ECD consists of SEQ ID NO: 18. In some embodiments, the CD16a ECD comprises the CD16a signal peptide and comprises amino acids 1-208 of SEQ ID NO: 12. In some embodiments, the CD16a ECD comprises the CD16a signal peptide and consists of amino acids 1-208 of SEQ ID NO: 12. In some embodiments, the CD16a ECD comprises the CD16a signal peptide and comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDVYIPKATL KDSGSYFCRGLFGSKNVSSETVNITITQGLAVSTISSFFPPGYQ (SEQ ID NO: 19). In some embodiments, the CD16a ECD comprises the CD16a signal peptide and consists of SEQ ID NO: 19. In some embodiments, the CD16b ECD comprises amino acids 17-208 of SEQ ID NO: 13. In some embodiments, the CD16b ECD consists of amino acids 17-208 of SEQ ID NO: 13. In some embodiments, the CD16b ECD comprises the amino acid sequence GMRTEDLPKAVVFLEPQWYSVLEKDSVTLKCQGAYSPEDNSTQWFHNESLISSQA SSYFIDAATVNDSGEYRCQTNLSTLSDPVQLEVHIGWLLLQAPRWVFKEEDPIHLR CHSWKNTALHKVTYLQNGKDRKYFHHNSDVHIPKATLKDSGSYFCRGLVGSKNV SSETVNITITQGLAVSTISSFSPPGYQ (SEQ ID NO: 20). In some embodiments, the CD16b ECD consists of SEQ ID NO: 20. In some embodiments, the CD16b ECD comprises the CD16a signal peptide and comprises amino acids 1-208 of SEQ ID NO: 13. In some embodiments, the CD16b ECD comprises the CD16b signal peptide and consists of amino acids 1-208 of SEQ ID NO: 13. In some embodiments, the CD16b ECD comprises the CD16a signal peptide and comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDVHIPKATL KDSGSYFCRGLVGSKNVSSETVNITITQGLAVSTISSFSPPGYQ (SEQ ID NO: 21). In some embodiments, the CD16b ECD comprises the CD16a signal peptide and consists of SEQ ID NO: 21.

In some embodiments, the CD16a ECD comprises amino acids 17-208 of SEQ ID NO: 14. In some embodiments, the CD16a ECD consists of amino acids 17-208 of SEQ ID NO: 14. In some embodiments, the CD16a ECD comprises the amino acid sequence GMRTEDLPKAVVFLEPQWYRVLEKDSVTLKCQGAYSPEDNSTQWFHNESLISSQA SSYFIDAATVDDSGEYRCQTNLSTLSDPVQLEVHIGWLLLQAPRWVFKEEDPIHLR CHSWKNTALHKVTYLQNGKGRKYFHHNSDFYIPKATLKDSGSYFCRGLFGSKNV SSETVNITITQGLAVPTISSFFPPGYQ (SEQ ID NO: 22). In some embodiments, the CD16a ECD consists of SEQ ID NO: 22. In some embodiments, the CD16a ECD comprises the CD16a signal peptide and comprises amino acids 1-208 of SEQ ID NO: 14. In some embodiments, the CD16a ECD comprises the CD16a signal peptide and consists of amino acids 1-208 of SEQ ID NO: 14. In some embodiments, the CD16a ECD comprises the CD16a signal peptide and comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDFYIPKATL KDSGSYFCRGLFGSKNVSSETVNITITQGLAVPTISSFFPPGYQ (SEQ ID NO: 23). In some embodiments, the CD16a ECD comprises the CD16a signal peptide and consists of SEQ ID NO: 23. In some embodiments, the CD16b ECD comprises amino acids 17-208 of SEQ ID NO: 15. In some embodiments, the CD16b ECD consists of amino acids 17-208 of SEQ ID NO: 15. In some embodiments, the CD16b ECD comprises the amino acid sequence GMRTEDLPKAVVFLEPQWYSVLEKDSVTLKCQGAYSPEDNSTQWFHNESLISSQA SSYFIDAATVNDSGEYRCQTNLSTLSDPVQLEVHIGWLLLQAPRWVFKEEDPIHLR CHSWKNTALHKVTYLQNGKDRKYFHHNSDFHIPKATLKDSGSYFCRGLVGSKNV SSETVNITITQGLAVPTISSFSPPGYQ (SEQ ID NO: 24). In some embodiments, the CD16b ECD consists of SEQ ID NO: 24. In some embodiments, the CD16b ECD comprises the CD16a signal peptide and comprises amino acids 1-208 of SEQ ID NO: 15. In some embodiments, the CD16b ECD comprises the CD16b signal peptide and consists of amino acids 1-208 of SEQ ID NO: 15. In some embodiments, the CD16b ECD comprises the CD16a signal peptide and comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDFHIPKATL KDSGSYFCRGLVGSKNVSSETVNITITQGLAVPTISSFSPPGYQ (SEQ ID NO: 25). In some embodiments, the CD16b ECD comprises the CD16a signal peptide and consists of SEQ ID NO: 25.

In some embodiments, the CD16a ECD comprises amino acids 17-208 of SEQ ID NO: 16. In some embodiments, the CD16a ECD consists of amino acids 17-208 of SEQ ID NO: 16. In some embodiments, the CD16a ECD comprises the amino acid sequence GMRTEDLPKAVVFLEPQWYRVLEKDSVTLKCQGAYSPEDNSTQWFHNESLISSQA SSYFIDAATVDDSGEYRCQTNLSTLSDPVQLEVHIGWLLLQAPRWVFKEEDPIHLR CHSWKNTALHKVTYLQNGKGRKYFHHNSDVYIPKATLKDSGSYFCRGLFGSKNV SSETVNITITQGLAVPTISSFFPPGYQ (SEQ ID NO: 26). In some embodiments, the CD16a ECD consists of SEQ ID NO: 26. In some embodiments, the CD16a ECD comprises the CD16a signal peptide and comprises amino acids 1-208 of SEQ ID NO: 16. In some embodiments, the CD16a ECD comprises the CD16a signal peptide and consists of amino acids 1-208 of SEQ ID NO: 16. In some embodiments, the CD16a ECD comprises the CD16a signal peptide and comprises the amino acid sequence MWQLLLPTALLLLVSAGMRTEDLPKAVVFLEPQWYRVLEKDSVTLKCQGAYSPE DNSTQWFHNESLISSQASSYFIDAATVDDSGEYRCQTNLSTLSDPVQLEVHIGWLL LQAPRWVFKEEDPIHLRCHSWKNTALHKVTYLQNGKGRKYFHHNSDVYIPKATL KDSGSYFCRGLFGSKNVSSETVNITITQGLAVPTISSFFPPGYQ (SEQ ID NO: 27). In some embodiments, the CD16a ECD comprises the CD16a signal peptide and consists of SEQ ID NO: 27. In some embodiments, the CD16b ECD comprises amino acids 17-208 of SEQ ID NO: 17. In some embodiments, the CD16b ECD consists of amino acids 17-208 of SEQ ID NO: 17. In some embodiments, the CD16b ECD comprises the amino acid sequence GMRTEDLPKAVVFLEPQWYSVLEKDSVTLKCQGAYSPEDNSTQWFHNESLISSQA SSYFIDAATVNDSGEYRCQTNLSTLSDPVQLEVHIGWLLLQAPRWVFKEEDPIHLR CHSWKNTALHKVTYLQNGKDRKYFHHNSDVHIPKATLKDSGSYFCRGLVGSKNV SSETVNITITQGLAVPTISSFSPPGYQ (SEQ ID NO: 28). In some embodiments, the CD16b ECD consists of SEQ ID NO: 28. In some embodiments, the CD16b ECD comprises the CD16a signal peptide and comprises amino acids 1-208 of SEQ ID NO: 17. In some embodiments, the CD16b ECD comprises the CD16b signal peptide and consists of amino acids 1-208 of SEQ ID NO: 17. In some embodiments, the CD16b ECD comprises the CD16a signal peptide and comprises the amino acid sequence MWQLLLPTALLLLVSAGMRTEDLPKAVVFLEPQWYSVLEKDSVTLKCQGAYSPE DNSTQWFHNESLISSQASSYFIDAATVNDSGEYRCQTNLSTLSDPVQLEVHIGWLL LQAPRWVFKEEDPIHLRCHSWKNTALHKVTYLQNGKDRKYFHHNSDVHIPKATL KDSGSYFCRGLVGSKNVSSETVNITITQGLAVPTISSFSPPGYQ (SEQ ID NO: 29). In some embodiments, the CD16b ECD comprises the CD16a signal peptide and consists of SEQ ID NO: 29.

In some embodiments, the CD16 fragment is comprised within a chimeric protein. In some embodiments, the chimeric protein is a fusion protein. In some embodiments, the chimeric protein is an artificial protein. In some embodiments, the protein is a receptor. CD16 fusion proteins capable of inducing ADCC and enhancing NK cell anti-tumor efficacy are disclosed for example in Meng et al., “Leveraging CD16 fusion receptors to remodel the immune response for enhancing anti-tumor immunotherapy in iPSC-derived NK cells”, 2023, Journal of Hematology & Oncology volume 16, Article number: 62, the contents of which are hereby incorporated by reference in their entirety.

In some embodiments, the chimeric protein comprises a hydrophobic domain. In some embodiments, a hydrophobic domain is a transmembrane domain. In some embodiments, a hydrophobic domain is a domain capable of inserting into a cellular membrane. In some embodiments, a hydrophobic domain is a domain capable of being inserted into a cellular membrane. In some embodiments, cellular membrane is a plasma membrane. In some embodiments, the CD16 fragment is N-terminal to the hydrophobic domain. In some embodiments, the CD16 fragment is C-terminal to the hydrophobic domain. The nine hydrophobic amino acids are glycine (G), alanine (A), valine (V), leucine (L), isoleucine (I), proline (P), phenylalanine (F), methionine (M), and tryptophan (W). In some embodiments, a hydrophobic domain comprises at least 6, 7, 8, 9, 10, 11, 12, 13, 14, 15, 16, 17, 18, 19 or 21 amino acids. Each possibility represents a separate embodiment of the invention. In some embodiments, a hydrophobic domain comprises at least 10 amino acids. In some embodiments, a hydrophobic domain comprises at least 20 amino acids. In some embodiments, a hydrophobic domain comprises at least 21 amino acids. In some embodiments, a hydrophobic domain comprises at least 50, 55, 60, 65 or 70% hydrophobic amino acids. Each possibility represents a separate embodiment of the invention. In some embodiments, a hydrophobic domain comprises at least 60% hydrophobic amino acids. In some embodiments, a hydrophobic domain comprises at least 70% hydrophobic amino acids. In some embodiments, a hydrophobic domain comprises at least 5, 6, 7, 8 or 9 consecutive hydrophobic amino acids. Each possibility represents a separate embodiment of the invention. In some embodiments, a hydrophobic domain comprises at least 7 consecutive hydrophobic amino acids. In some embodiments, a hydrophobic domain comprises at least 9 consecutive hydrophobic amino acids.

In some embodiments, the hydrophobic domain comprises the transmembrane domain of CD16a. In some embodiments, the hydrophobic domain consists of the transmembrane domain of CD16a. In some embodiments, the transmembrane domain of CD16a comprises amino acids 209-229 of SEQ ID NO: 2. In some embodiments, the transmembrane domain of CD16a consists of amino acids 209-229 of SEQ ID NO: 2. In some embodiments, the transmembrane domain of CD16a comprises the amino acid sequence VSFCLVMVLLFAVDTGLYFSV (SEQ ID NO: 9). In some embodiments, the transmembrane domain of CD16a consists of SEQ ID NO: 9.

In some embodiments, the chimeric protein comprises an intracellular domain. In some embodiments, the intracellular domain is C-terminal to the hydrophobic domain. In some embodiments, the intracellular domain is N-terminal to the hydrophobic domain. In some embodiments, the CD16 fragment and the intracellular domain are separated by the hydrophobic domain. In some embodiments, the intracellular domain is a signaling domain.

In some embodiments, the intracellular domain comprises at least one signaling domain. In some embodiments, the signaling domain is an activating domain. In some embodiments, the signaling domain is an immune cell signaling domain. In some embodiments, the signaling domain is an immune cell activating domain. In some embodiments, the signaling domain is an ITAM domain. In some embodiments, the intracellular domain comprises at least 1, 2, 3, 4, 5 or 6 ITAM domains. Each possibility represents a separate embodiment of the invention. In some embodiments, the activating domain is selected from an immune cell activating protein. In some embodiments, the activating protein is selected from 2B4 (CD244), DAP10 (HCST), CD3zeta (CD3Z/CD247) and FCRgamma (FCER1G).

In some embodiments, the intracellular domain comprises a 2B4 signaling domain. In some embodiments, the 2B4 signaling domain comprises amino acids 251-370 of UniProt sequence Q9BZW8. In some embodiments, the 2B34 signaling domain comprises or consists of

(SEQ ID NO: 4)
WRRKRKEKQSETSPKEFLTIYEDVKDLKTRRNHEQEQTFPGGGST
IYSMIQSQSSAPTSQEPAYTLYSLIQPSRKSGSRKRNHSPSFNST
IYEVIGKSQPKAQNPARLSRKELENFDVYS.

In some embodiments, the intracellular domain comprises a DAP10 signaling domain. In some embodiments, the DAP10 signaling domain comprises amino acids 70-93 of UniProt sequence Q9UBK5. In some embodiments, the DAP10 signaling domain comprises or consists of LCARPRRSPAQEDGKVYINMPGRG (SEQ ID NO: 5).

In some embodiments, the intracellular domain comprises a CD3Z signaling domain. In some embodiments, the CD3Z signaling domain comprises amino acids 52-164 of UniProt sequence P20963. In some embodiments, the CD3Z signaling domain comprises or consists of

(SEQ ID NO: 6)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPR.

In some embodiments, the intracellular domain comprises an FCER1G signaling domain. In some embodiments, the FCER1G signaling domain comprises amino acids 45-86 of UniProt sequence P30273. In some embodiments, the FCER1G signaling domain comprises or consists of

(SEQ ID NO: 7)
RLKIQVRKAAITSYEKSDGVYTGLSTRNQETYETLKHEKPPQ.

In some embodiments, the intracellular domain comprises a plurality of signaling domains. In some embodiments, the at least one signaling domain is a plurality of signaling domains. In some embodiments, the plurality comprises at least two different signaling domains. In some embodiments, the intracellular domain comprises a 2B4 domain, a DAP10 domain and a CD3Z domain. In some embodiments, the 2B4 domain is N-terminal to the DAP10 domain and the DAP10 domain is N-terminal to the CD3Z domain. In some embodiments, the plurality of domains are contiguous to each other. In some embodiments, the plurality of domains are separated by spacers. In some embodiments, the intracellular domain consists of the at least one signaling domain. In some embodiments, the hydrophobic domain is contiguous to the plurality of signaling domains. In some embodiments, the hydrophobic domain is separated from the at least one signaling domain by a spacer.

As used herein, the term “CAR” refers to an engineered receptor, which has specificity for at least one protein of interest. Chimeric antigen receptors (CARs) are well known in the art, and CARs specific to CD19 are also well known. The polypeptide contains an extracellular CD-19-binding domain, a transmembrane domain and an intracellular signaling domain. First generation CARs make use of a CD3Z signaling domain, but later generation CARs can also have additional signaling/costimulatory domains (i.e., second generation CARs containing the CD28 costimulatory domain or 41BB costimulatory domain, and third generation CARs containing both). The extracellular antigen binding domain can contain a single chain antibody against the target (e.g., anti-CD19 ScFv).

Commercially available anti-CD19 CARs can be purchased for example from BPS Bioscience (catalog #78601), Creative Biolabs (catalog #: CAR-YF001, CAR-YF002, CAR-YF-019, CAR-YF-020, CAR-YF-025, CAR-YF-026, CAR-YF-090, CAR-YF-091, CAR-ZP021, CAR-ZP024, CAR-ZP-047, CAR-ZP-050, CAR-ZP3082, CAR-YF418, CAR-YF410) and ProMab Biotechnologies (catalog #: PM-CAR1001-1004, 1006-1007, 1009-1010, 1035, 1042-1043, 10481050, 1054-1055, 1066-1067, 1071, 1087-1088, 1090) among many others.

In some embodiments, the anti-CD19 CAR comprises an anti-CD19 single chain variable fragment (scFV). In some embodiments, the scFV is a single chain antibody. In some embodiments, the extracellular domain of the CAR comprises the scFV. In some embodiments, the extracellular domain of the CAR consists of the scFV. In some embodiments, the scFV is the FMC63 anti-CD19 scFV. The FMC63 antibody was first disclosed in Nicholson et al., “Construction and characterisation of a functional CD19 specific single chain Fv fragment for immunotherapy of B lineage leukaemia and lymphoma”, Mol Immunol. 1997 November-December; 34(16-17):1157-65, the contents of which are hereby incorporated by reference in their entirety. A CAR comprising the FMC63 scFV was first described in Kochenderfer et al., “Construction and Pre-clinical Evaluation of an Anti-CD19 Chimeric Antigen Receptor”, J. Immunotherapy, 2009 September; 32(7):689-702, the contents of which are hereby incorporated by reference in its entirety. In some embodiments, the CAR comprises a CD28 costimulatory domain. In some embodiments, the CAR comprises a CD3zeta signaling domain. In some embodiments, the CAR is FMC63-28-3z. The nucleic acid sequence encoding FMC63-28-3z is available from Addgene in catalog number 135991.

In some embodiments, the CD16 or fragment thereof is on the plasma membrane of the cell. In some embodiments, the anti-CD19 CAR is on the plasma membrane of the cell. In some embodiments, the CD16 or fragment thereof is in the plasma membrane of the cell. In some embodiments, the anti-CD19 CAR is in the plasma membrane of the cell. In some embodiments, the CD16 or fragment thereof is transmembrane. In some embodiments, the anti-CD19 CAR is transmembrane.

In some embodiments, the target cell disclosed herein is a cancer cell. In some embodiments, the cancer cell is in a subject. In some embodiments, the cancer cell expresses CD48. In some embodiments, the cancer is a CD48 cancer. In some embodiments, the cancer is a CD48 positive cancer. In some embodiments, the cancer is a CD48 expressing cancer. In some embodiments, the cancer is a hematopoietic cancer. In some embodiments, a CD48 expressing cancer is a hematopoietic cancer. In some embodiments, a CD48 expressing cancer is a blood cancer. In some embodiments, a CD48 expressing cancer is selected from lymphoma, myeloma and leukemia. In some embodiments, a CD48 expressing cancer is selected from lymphoma and myeloma.

In some embodiments, the cell disclosed herein, being a non-proliferating cell of an immortalized NK cell line, expresses a transmembrane receptor for CD48. In some embodiments, the cell expresses a transmembrane protein that binds CD48. As used herein, the terms “cluster of differentiation 48 (CD48)”, “CD48 antigen”, “B-lymphocyte activation marker (BLAST-1)”, and “signaling lymphocytic activation molecule 2 (SLAMF2)” are used interchangeably and encompass a cell surface protein encoded by the CD48 gene. CD48 protein is a known receptor, being a member of the CD2 subfamily of the immunoglobulin superfamily (IgSF). The human CD48 gene is provided in Entrez gene #962, its mRNA sequence can be found in RefSeq NM 001778 and NM_001256030 and the amino acid sequence can be found in Uniprot #P09326. In some embodiments, the transmembrane receptor for CD48 is mammalian. In some embodiments, the transmembrane receptor for CD48 is human.

In some embodiments, the CD48 receptor is a cell surface protein. In some embodiments, the receptor for CD48 is a transmembrane protein. In some embodiments, the receptor for CD48 is a protein expressed by an NK cell. In some embodiments, the receptor for CD48 mediates NK cytotoxic activity. In some embodiments, the receptor for CD48 is 2B4. In some embodiments, there is provided a pharmaceutical composition comprising a non-proliferating cell of an immortalized NK cell line, being characterized by: (i) exogenously expressing the lncRNA NeST; and (ii) expressing the receptor 2B4.

The terms “2B4”, “Cluster of differentiation 244 (CD244)”, “NAIL”, “NKR2B4”, “Nmrk”, and “SLAMF4”, are used interchangeably and refer to a protein encoded by the CD244 gene. In some embodiments, 2B4 is an NK cell receptor. It should be noted that the terms “receptor” and “ligand” as disclosed herein can be used interchangeably, depending on the referent cell being activated. In some embodiments, CD48 is a ligand expressed by the target cell disclosed herein. In some embodiments, 2B4 is a receptor expressed by the non-proliferating cell of the immortalized NK cell line, as disclosed herein. In some embodiments, binding between CD48, expressed by the target cell, and 2B4, expressed by the non-proliferating cell, activates the non-proliferating cell. In some embodiments, activation comprises increased cytotoxic activity against the target cell. In some embodiments, activation comprises increased inflammatory activity against the target cell. In some embodiments, activation comprises increased cytotoxic activity and increased inflammatory activity. In some embodiments, the cytotoxic activity is mediated by the inflammatory activity.

In some embodiments, the non-proliferating cell of the immortalized NK cell line is characterized by increased cytotoxic activity against a target cell. In some embodiments, the non-proliferating cell is characterized by increased inflammatory activity. In some embodiments, the non-proliferating cell is characterized by increased cytotoxic activity against a target cell and increased inflammatory activity. In some embodiments, increased inflammatory activity comprises increased pro-inflammatory cytokine expression and/or secretion. In some embodiment, increased cytotoxic activity comprises increased killing activity of the target cell. In some embodiments, the pro-inflammatory cytokine is selected from: interferon-γ (IFN-γ), tumor necrosis factor-α (TNF-α), granulocyte macrophage colony-stimulating factor (GM-CSF), or any combination thereof. In some embodiments, the pro-inflammatory cytokine comprises IFN-γ. In some embodiments, the pro-inflammatory cytokine is IFN-γ.

In some embodiments, increased cytotoxic activity, inflammatory activity, or both, is as compared to a control cell. In some embodiments, the control cell comprises a cell of an immortalized NK cell line, not exogenously expressing the lncRNA NeST. In some embodiments, the control cell comprises a cell of an immortalized NK cell line that is not proliferating. In some embodiments, a control cell comprises a non-proliferating cell of an immortalized NK cell line, not exogenously expressing the lncRNA NeST. In some embodiments, a control cell comprises a non-proliferating cell of an immortalized NK cell line, expressing endogenous levels of the lncRNA NeST. In some embodiments, a control cell comprises an irradiated cell of an immortalized NK cell line, expressing endogenous levels of the lncRNA NeST. In some embodiments, the control cell is irradiated with the same irradiation dose as the non-proliferating cell of the immortalized NK cell line disclosed herein.

In some embodiments, binding between CD48, expressed by the target cell, and 2B4, expressed by the non-proliferating cell, increases the expression, secretion, or both, of IFN-7 by at least 10, 20, 30, 40, 50, 60, 70, 80, 90 or 100%. Each possibility represents a separate embodiment of the present invention. In some embodiments, binding between CD48, expressed by the target cell, and 2B4, expressed by the non-proliferating cell, increases the expression, secretion, or both, of IFN-γ by at least 10%. In some embodiments, increased expression, secretion, or both, of IFN-γ is by 10% to 300%, by 10% to 250%, by 10% to 200%, by 10% to 150%, by 10% to 100%, by 20% to 300%, by 20% to 250%, by 20% to 200%, by 20% to 150%, by 20% to 100%, by 30% to 300%, by 30% to 250%, by 30% to 200%, by 30% to 150%, by 30% to 100%, by 40% to 300%, by 40% to 250%, by 40% to 200%, by 40% to 150%, by 40% to 100%, by 10% to 150%, by 10% to 140%, by 10% to 130%, by 10% to 120%, by 10% to 110%, by 10% to 100%, by 10% to 90%, by 10% to 80%, by 20% to 150%, by 20% to 140%, by 20% to 130%, by 20% to 120%, by 20% to 110%, by 20% to 100%, by 20% to 90%, by 20% to 80%, by 30% to 150%, by 30% to 140%, by 30% to 130%, by 30% to 120%, by 30% to 110%, by 30% to 100%, by 30% to 90%, by 30% to 80%, by 30% to 150%, by 30% to 140%, by 30% to 130%, by 40% to 120%, by 40% to 110%, by 40% to 100%, by 40% to 90%, or by 40% to 80%. Each possibility represents a separate embodiment of the present invention.

In some embodiments, binding between CD48, expressed by the target cell, and 2B4, expressed by the non-proliferating cell, increases the death of the target cell. In some embodiments, the death of the target cell comprises a programmed cell death. In some embodiments, the death is selected from apoptosis, autophagic cell death, necrosis, or any combination thereof. In some embodiments, the death comprises increased lysis of the cell.

In some embodiments, increased cell death comprises increased necrosis. In some embodiments, increased cell death is by at least 10, 20, 30, 40, 50, 60, 70, 80, 90 or 100%. In some embodiments, increased cell death is by at least 10%. In some embodiments, increased cell death by 50% to 300%, by 50% to 250%, by 50% to 200%, by 50% to 150%, by 60% to 300%, by 60% to 250%, by 60% to 200%, by 60% to 150%, by 70% to 300%, by 70% to 250%, by 70% to 200%, by 70% to 150%, by 80% to 300%, by 80% to 250%, by 80% to 200%, by 80% to 150%, by 90% to 300%, by 90% to 250%, by 90% to 200%, by 90% to 150%, by 50% to 140%, by 50% to 130%, by 50% to 120%, by 50% to 110%, by 60% to 140%, by 60% to 130%, by 60% to 120%, by 60% to 110%, by 70% to 140%, by 70% to 130%, by 70% to 120%, by 70% to 110%, by 80% to 140%, by 80% to 130%, by 80% to 120%, by 80% to 110%, by 90% to 140%, by 90% to 130%, by 90% to 120%, or by 90% to 110%. Each possibility represents a separate embodiment of the present invention.

Methods for detection of pro-inflammatory cytokines (e.g., IFN-7) expression, secretion, or both, are well-known in the art, including immunoassays, ELISA, flow cytometry, and Western-Blot. Methods for detecting cell proliferation or death are well known. Non-limiting examples include colorimetric based assays (e.g., XTT or MTT proliferation assay).

In some embodiments, the method is an in vitro method. In some embodiments, the method is an ex vivo method. In some embodiments, the method is an in vivo method. In some embodiments, binding between CD48, expressed by the target cell, and 2B4, expressed by the non-proliferating cell, is in-vitro. In some embodiments, binding between CD48 and 2B4 is ex-vivo. In some embodiments, binding between CD48, expressed by the target cell, and 2B4, expressed by the non-proliferating cell, is in-vivo. In some embodiments, binding between CD48, expressed by the target cell, and 2B4, expressed by the non-proliferating cell, is in a subject in need thereof. In some embodiments, the subject is mammalian. In some embodiments, the subject is a human. In some embodiments, the subject suffers from cancer. In some embodiments, the subject is in need of a method of treatment of the invention.

In some embodiments, the cell disclosed herein comprises a nucleic acid molecule comprising a polynucleotide sequence encoding the lncRNA NeST. In some embodiments, the lncRNA NeST is mammalian. In some embodiments, the lncRNA NeST is human. In some embodiments, the nucleic acid molecule encoding NeST comprises a plasmid or an expression vector. In some embodiments, the nucleic acid molecule encoding NeST comprises a nucleotide sequence as set forth in SEQ ID NO:1. In some embodiments, exogenous expression comprises exogenous expression of a non-genomic nucleic acid molecule comprising a polynucleotide sequence encoding NeST. In some embodiments, exogenous expression comprises integration into the genome of the cell of an exogenous copy of NeST.

In some embodiments, the cell disclosed herein comprises a nucleic acid molecule comprising a polynucleotide sequence encoding the CD16 or fragment thereof. In some embodiments, the cell disclosed herein comprises a nucleic acid molecule comprising a polynucleotide sequence encoding the CAR. In some embodiments, the nucleic acid molecule is a plasmid. In some embodiment, the plasmid is an expression plasmid. In some embodiments, the nucleic acid molecule is a vector. In some embodiments, the vector is an expression vector. In some embodiments, exogenous expression comprises integration into the genome of the cell of an exogenous sequence encoding the CD16 or fragment thereof. In some embodiments, exogenous expression comprises integration into the genome of the cell of an exogenous sequence encoding the CAR.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectious origin, amplification products, modified nucleic acids, plasmidical or organelle nucleic acids and artificial nucleic acids such as oligonucleotides.

As used herein, the term “encoding” refers to a molecule comprising a DNA sequence which can be transcribed into an RNA sequence. In some embodiments, the RNA is an lncRNA. In some embodiments, the RNA is an mRNA. In some embodiments, the RNA can be translated into the encoded protein or a molecule comprising the RNA sequence can be translated into the encoded protein. In some embodiments, the molecule is a DNA molecule. In some embodiments, the molecule is an RNA molecule. In some embodiments, the DNA is cDNA. In some embodiments, the molecule is a DNA/RNA hybrid. In some embodiments, the molecule comprises non-naturally occurring nucleotides.

Expression of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. In some embodiments, the vector comprises a lentiviral vector. In some embodiments, expression of the polynucleotide encoding NeST in the cell is by transfecting with a lentiviral vector. In some embodiments, expression of the polynucleotide encoding CD16 or a fragment thereof in the cell is by transfecting with a lentiviral vector. In some embodiments, expression of the polynucleotide encoding the CAR in the cell is by transfecting with a lentiviral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoter may be a viral promoter.

In some embodiments, the polynucleotide is operably linked to a transcription regulatory element. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the transcription regulatory element is a promoter. In some embodiments, the promoter is operably linked to the polynucleotide of the invention. In some embodiments, the promoter is a heterologous promoter. In some embodiments, the promoter is the endogenous promoter.

In some embodiments, the promoter is a constitutive promoter. In some embodiments, the promoter is active in NK cells. In some embodiments, the promoter is an NK cell specific promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. [096] The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-lMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

In some embodiments, the cell of the immortalized NK cell line exogenously expressing NeST, is an irradiated cell. In some embodiments, the irradiated cell is irradiated with ionizing radiation. In some embodiments, ionizing radiation comprises γ radiation. As used herein, the term “ionizing radiation” refers to subatomic particles or electromagnetic waves that have sufficient energy to ionize atoms or molecules by detaching electrons from them. As used herein, the term “γ radiation” encompasses a known penetrating form of electromagnetic radiation arising from the radioactive decay of atomic nuclei. In some embodiments, γ radiation comprises the shorter wavelength electromagnetic waves, compared to other electromagnetic waves.

In some embodiments, irradiation is with a dose sufficient to render the cell of the immortalized NK cell line non-proliferative. In some embodiments, irradiation is with an ionizing radiation dose above 1000 cGY. In some embodiments, irradiation is with an ionizing radiation dose above 2000 cGY. In some embodiments, irradiation is with a dose that does not impair the cytotoxic activity of the cell against the target cell. In some embodiments, irradiation is with a dose that does not reduce IFN-γ expression, secretion, or both. In some embodiments, irradiation is with a dose that does not reduce the killing of the target cell. In some embodiments, does not reduce is does not substantially reduce. In some embodiments, does not reduce is does not significantly reduce. In some embodiments, the irradiation dose is between 1000 cGY and 10000 cGY, between 1000 cGY and 9000 cGY, between 1000 cGY and 8000 cGY, between 1000 cGY and 7000 cGY, between 1000 cGY and 6000 cGY, between 1500 cGY and 10000 cGY, between 1500 cGY and 9000 cGY, between 1500 cGY and 8000 cGY, between 1500 cGY and 7000 cGY, or between 1500 cGY and 6000 cGY. Each possibility represents a separate embodiment of the present invention. In some embodiments, irradiation is with an ionizing radiation dose between 1500 cGY and 6000 cGY, between 1500 cGY and 5500 cGY, between 1500 cGY and 5000 cGY, between 1500 cGY and 4500 cGY, between 1500cGY and 4000cGY, between 1500cGY and 3500 cGY, between 1500 cGY and 3000 cGY, between 2000 cGY and 6000cGY, between 2000 cGY and 5500cGY, between 2000 cGY and 5000 cGY, between 2000 cGY and 4500 cGY, between 2000 cGY and 4000 cGY, between 2000 cGY and 3500 cGY, or between 2000 cGY and 3000 cGY. Each possibility represents a separate embodiment of the present invention. In some embodiments, the irradiation dose is about 2000 cGy. In some embodiments, the irradiation dose is between 2000 and 3000 cGy. In some embodiments, the irradiation dose is between 2000 and 5000 cGy.

In some embodiments, the pharmaceutical composition disclosed herein is for use in treating a CD48 expressing cancer in a subject in need thereof. In some embodiments, CD48 expressing cancer is a CD48 positive cancer.

By another aspect, there is provided a method of treating a CD48 expressing cancer in a subject in need thereof. In some embodiments, the method comprises administering to the subject the pharmaceutical composition disclosed herein. In some embodiments, the method comprises administering to the subject an effective amount of the pharmaceutical composition. In some embodiments, effective is therapeutically effective. In some embodiments, effective is effective in treating cancer.

As used herein, the term “subject” refers to any subject, including a mammalian subject, for whom therapy is desired, for example, a human. In some embodiments, the subject is a human subject. In some embodiments, the subject suffers from cancer. In some embodiments, the subject is confirmed to suffer from CD48 expressing cancer. In some embodiments, the method further comprises selecting a subject suffering from CD48 expressing cancer. In some embodiments, the subject is confirmed to suffer from CD19 expressing cancer. In some embodiments, the method further comprises selecting a subject suffering from CD19 expressing cancer. In some embodiments, the method further comprises receiving a sample from the subject. In some embodiments, the sample comprises cells. In some embodiments, the cells are cancer cells. In some embodiments, the method further comprises detecting CD48 expressing in the sample. In some embodiments, the detecting is detecting CD48 expression in the cells. In some embodiments, CD48 expression is CD48 surface expression. In some embodiments, the method further comprises detecting CD19 expressing in the sample. In some embodiments, the detecting is detecting CD19 expression in the cells. In some embodiments, CD19 expression is CD19 surface expression.

In some embodiments, a CD48 level above a predetermined threshold is indicative of a CD48 expressing cancer. In some embodiments, the threshold is the CD48 expression level in noncancerous cells. In some embodiments, the noncancerous cells are of the same cell type as the cancerous cells. In some embodiments, a CD48 level above a predetermined threshold comprises increased CD48 surface protein level on the cancer cell of the subject.

In some embodiments, increased is as compared to noncancerous cells. In some embodiments, the CD48 levels are in the cancer cells. In some embodiments, CD48 expression in a cancer cell is indicative of a CD48 expressing cancer. In some embodiments, the method further comprises selecting a subject whose sample comprises CD48 expressing cancer cells.

In some embodiments, a CD19 level above a predetermined threshold is indicative of a CD19 expressing cancer. In some embodiments, the threshold is the CD19 expression level in noncancerous cells. In some embodiments, the noncancerous cells are of the same cell type as the cancerous cells. In some embodiments, a CD19 level above a predetermined threshold comprises increased CD19 surface protein level on the cancer cell of the subject.

In some embodiments, increased is as compared to noncancerous cells. In some embodiments, the CD19 levels are in the cancer cells. In some embodiments, CD19 expression in a cancer cell is indicative of a CD19 expressing cancer. In some embodiments, the method further comprises selecting a subject whose sample comprises CD19 expressing cancer cells.

In some embodiments, the method further comprises a step of determining the expression level of CD48. In some embodiments, expression is surface expression. In some embodiments, expression comprises secretion. In some embodiments, CD48 levels are within the cancer. In some embodiments, CD48 levels are within the tumor. In some embodiments, CD48 levels are within the tumor microenvironment (TME). In some embodiments, an expression level of CD48 is above a predetermined threshold indicates the subject is suitable for treatment with the pharmaceutical composition of the invention or by a method of the invention. In some embodiments, CD48 expression in cancer cells from the subject indicates the subject is suitable for treatment with the pharmaceutical composition of the invention or by a method of the invention.

In some embodiments, the method further comprises a step of determining the expression level of CD19. In some embodiments, expression is surface expression. In some embodiments, CD19 levels are within the cancer. In some embodiments, CD19 levels are within the tumor. In some embodiments, CD19 levels are within the tumor microenvironment (TME). In some embodiments, an expression level of CD19 is above a predetermined threshold indicates the subject is suitable for treatment with the pharmaceutical composition of the invention or by a method of the invention. In some embodiments, CD19 expression in cancer cells from the subject indicates the subject is suitable for treatment with the pharmaceutical composition of the invention or by a method of the invention.

In some embodiments, an expression level of CD48 below or equal to a predetermined threshold, indicates the subject is not suitable for treatment with the pharmaceutical composition of the invention or by the method of the invention. In some embodiments, absence of CD48 expression from cancer cells of the subject indicates the subject is not suitable for treatment with the pharmaceutical composition of the invention or by the method of the invention.

In some embodiments, expression level of CD19 below or equal to a predetermined threshold, indicates the subject is not suitable for treatment with the pharmaceutical composition of the invention or by the method of the invention. In some embodiments, absence of CD19 expression from cancer cells of the subject indicates the subject is not suitable for treatment with the pharmaceutical composition of the invention or by the method of the invention.

By another aspect, there is provided a method for selecting a subject being suitable for treatment with the composition of the invention, comprising the steps of: (a) determining the expression of CD48 in cancer cells of the subject, and (b) administering to a subject comprising cancer cells expressing CD48 a therapeutically effective amount of the pharmaceutical composition of the invention.

By another aspect, there is provided a method for selecting a subject being suitable for treatment with the composition of the invention, comprising the steps of: (a) determining the expression of CD19 in cancer cells of the subject, and (b) administering to a subject comprising cancer cells expressing CD19 a therapeutically effective amount of the pharmaceutical composition of the invention.

In some embodiments, the determining step is performed in the subject or in a sample derived or obtained from the subject. In some embodiments, the sample comprises a bodily fluid, cell, tissue, biopsy, organ, or a combination thereof. In some embodiments, the sample is selected from: peripheral blood, plasma, serum, tumor biopsy, tumor fluid, or any combination thereof. In some embodiments, the sample is a biopsy. In some embodiments, the sample is a blood sample. In some embodiments, the determining step is performed in vivo, ex vivo, or in vitro. In some embodiments, the determining step is performed ex vivo. In some embodiments, the method further comprises obtaining a sample from the subject. In some embodiments, the sample comprises cancer cells.

Methods and means for in vitro and/or ex vivo assays, as described herein, are common and would be apparent to one of ordinary skill in the art (e.g., flow cytometry, western blot, and immunohistochemistry).

In some embodiments, CD48 positive cancer comprises a solid tumor. In some embodiments, CD48 positive cancer comprises a liquid tumor. In some embodiments, a liquid tumor is a hematopoietic cancer. In some embodiments, CD48 positive cancer is a blood cancer. In some embodiments, a liquid tumor comprises blood cancer, bone marrow cancer, lymph node cancer, or any combination thereof. In some embodiments, a liquid tumor is selected from: leukemia, lymphoma, multiple myeloma, or any combination thereof. In some embodiments, a liquid tumor comprises lymphoma. In some embodiments, CD48 positive cancer is selected from lymphoma, myeloma and leukemia. In some embodiments, CD48 positive cancer is selected from lymphoma and myeloma. In some embodiments, the myeloma is multiple myeloma.

In some embodiments, CD19 positive cancer comprises a solid tumor. In some embodiments, CD19 positive cancer comprises a liquid tumor. In some embodiments, CD19 positive cancer is a B cell malignancy. In some embodiments, the B cell malignancy is selected from B-cell acute lymphoblastic leukemia (B-ALL), chronic lymphocytic leukemia (CLL), and B-cell non-Hodgkin lymphoma (B-NHL).

In some embodiments, CD48 positive cancer comprises at least one cancer type selected from: lymphoma, leukemia, multiple myeloma, hepatocellular carcinoma, glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, melanoma, or any combination thereof.

In some embodiments, CD48 positive cancer comprises a hematopoietic cancer. In some embodiments, CD48 positive cancer is a hematopoietic cancer. In some embodiments, the hematopoietic cancer is selected from: leukemia, lymphoma, multiple myeloma, or any combination thereof. In some embodiments, the hematopoietic cancer comprises leukemia.

In some embodiments, the hematopoietic cancer comprises lymphoma. In some embodiments, the CD48 positive cancer comprises lymphoma. In some embodiments, the CD48 positive cancer is lymphoma. In some embodiments, the pharmaceutical composition disclosed herein is for use in treating lymphoma in a subject in need thereof.

In some embodiments, the pharmaceutical composition comprises a therapeutically effective amount of non-proliferating cells. In some embodiments, therapeutically effective is in treating cancer. In some embodiments, cancer is CD48 expressing cancer. In some embodiments, cancer is CD19 expressing cancer. In some embodiments, there is provided a pharmaceutical composition comprising a therapeutically effective amount of non-proliferating cells as disclosed herein and a pharmaceutically acceptable carrier, excipient or adjuvant.

The term “pharmaceutically acceptable” means suitable for administration to a subject, e.g., a human. For example, the term “pharmaceutically acceptable” can mean approved by a regulatory agency of the Federal or a state government or listed in the U. S.

Pharmacopeia or other generally recognized pharmacopeia for use in animals, and more particularly in humans.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S.

Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum.

Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for administration to a subject. In some embodiments, the subject is a mammal. In some embodiments, the mammal is a human. In some embodiments, the human is in need of the administration. In some embodiments, the pharmaceutical composition is formulated for systemic administration. In some embodiments, systemic administration is administration to the bloodstream of a subject. In some embodiments, the pharmaceutical composition is formulated for intravenous administration. In some embodiments, the pharmaceutical composition is formulated for administration to the bloodstream.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal.

The dosage administered will be dependent upon the age, health, and weight of the recipient, kind of concurrent treatment, if any, frequency of treatment, and the nature of the effect desired.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

### Methods of Production

By another aspect there is provided a method for producing a non-proliferating NK cell, the method comprising:
(a) providing a cell of an immortalized NK cell line exogenously expressing a long non-coding RNA (lncRNA) NeST; and (b) irradiating the cell of step (a) with ionizing radiation; or,
(a) providing a cell of an immortalized NK cell line, (b) irradiating the cell of step (a) with ionizing radiation, and (c) exogenously expressing the lncRNA NeST in the irradiated cell of step (b);
   thereby, producing a non-proliferating NK cell.

By another aspect there is provided a method for producing a non-proliferating NK cell, the method comprising:
(a) providing a cell of an immortalized NK cell line exogenously expressing CD16 or an Fc-binding fragment thereof, and (b) irradiating the cell of step (a) with ionizing radiation; or,
(a) providing a cell of an immortalized NK cell line, (b) irradiating the cell of step (a) with ionizing radiation, and (c) exogenously expressing CD16 or an Fc-binding fragment thereof in the irradiated cell of step (b);
   thereby, producing a non-proliferating NK cell.

By another aspect there is provided a method for producing a non-proliferating NK cell, the method comprising:
(a) providing a cell of an immortalized NK cell line exogenously expressing an anti-CD19 CAR; and (b) irradiating the cell of step (a) with ionizing radiation; or,
(a) providing a cell of an immortalized NK cell line, (b) irradiating the cell of step (a) with ionizing radiation, and (c) exogenously expressing an anti-CD19 CAR in the irradiated cell of step (b);
   thereby, producing a non-proliferating NK cell.

In some embodiments, the method comprises: (a) providing a cell of an immortalized NK cell line exogenously expressing the lncRNA NeST; and, (b) irradiating the cell of step (a) with ionizing radiation. In some embodiments, the method comprises: (a) providing a cell of an immortalized NK cell line; (b) irradiating the cell of step (a) with ionizing radiation and (c) exogenously expressing the lncRNA NeST in the irradiated cell of step (b). In some embodiments, the method comprises: (a) providing a cell of an immortalized NK cell line exogenously expressing CD16 or an Fc-binding fragment thereof, and, (b) irradiating the cell of step (a) with ionizing radiation. In some embodiments, the method comprises: (a) providing a cell of an immortalized NK cell line; (b) irradiating the cell of step (a) with ionizing radiation and (c) exogenously expressing CD16 or an Fc-binding fragment thereof in the irradiated cell of step (b). In some embodiments, the method comprises: (a) providing a cell of an immortalized NK cell line exogenously expressing an anti-CD19 CAR; and, (b) irradiating the cell of step (a) with ionizing radiation. In some embodiments, the method comprises: (a) providing a cell of an immortalized NK cell line; (b) irradiating the cell of step (a) with ionizing radiation and (c) exogenously expressing an anti-CD19 CAR in the irradiated cell of step (b). In some embodiments, irradiating is with a dose sufficient to render the cell of the immortalized NK cell line non-proliferative.

In some embodiments, the method further comprises measuring proliferation of the cell after irradiation, and selecting a cell that does not proliferate. In some embodiments, the method further comprises measuring cytotoxicity of the cell after irradiation and selecting a cell that is still cytotoxic. In some embodiments, cytotoxic comprises secretion of at least one proinflammatory cytokine. In some embodiments, the selecting is selecting any cytotoxic cell. In some embodiments, the selecting is selecting the most cytotoxic cell. In some embodiments, the most is the top 50, 45, 40, 35, 30, 25, 20, 15, 10, or 5% of cells. Each possibility represents a separate embodiment of the invention. In some embodiments, the most is the top 25% of cells. In some embodiments, the selecting is selecting a cell that retains cytotoxicity at least 1, 2, 3, 4, 5, 6, 7, 8, 9, 10, 11, 12, 13, or 14 days after the irradiation. Each possibility represents a separate embodiment of the invention. In some embodiments, the selecting is selecting a cell that retains cytotoxicity at least 3 days after the irradiation. In some embodiments, retains is retains at least 95, 90, 85, 80, 75, 70, 65, 60, 55, 50, 45, 40, 35, 30, 25, 20, 15, or 10% of the cytotoxicity from before the irradiation. Each possibility represents a separate embodiment of the invention. In some embodiments, retains is retains at least 50% of the cytotoxicity from before the irradiation. In some embodiments, retaining cytotoxicity comprises retaining proinflammatory cytokine secretion. In some embodiments, retaining cytotoxicity comprises retaining cell killing. In some embodiments, cell killing is specific cell killing. In some embodiments, specific is specific to CD48.

In some embodiments, there is provided a pharmaceutical composition comprising a non-proliferating NK cell obtainable by the method disclosed herein. In some embodiments, there is provided a pharmaceutical composition comprising a non-proliferating NK cell produced by the method disclosed herein.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances, where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Cell handling: K562, BCBL1, YTS, P815, Jurkat, BJAB, RAJI, BW, 8866, EL-4 and 721.221 cells were maintained in RPMI (Sigma-Aldrich) supplemented with 10% fetal calf serum (Sigma-Aldrich), 2 mM glutamine (Biological Industries (BI)), 1 mM sodium pyruvate (BI), 1× nonessential amino acids (BI), 100 U/ml penicillin (BI), 0.1 mg/ml streptomycin (BI).

For NK-92 the same medium and conditions were used, but 200U/ml IL-2 (PeproTech) were added to the medium.

Transduction: The plasmid construct dsRed was modified with the NeST gene sequence GenBank: MK296539 and then lentivirally transduced. Lentiviruses were replicated in 293T cells utilizing Mirus TransIT-LT1 and two plasmids, Gag-pol and pMDG. After 48 h virus production, the supernatants were harvested and used for transducing the YTS cells. 200,000 cells were transduced at once through spinfection for 90 min at 1600 RPMI at 25° C. in a 96 U-plate. After 24 h the cells were resupended in virus-free RPMI medium and cultured further. dsRed contains the GFP gene, which was used to sort the successfully transduced cells.

Target staining: To verify the surface expression of CD48 on potential target cells FACS stainings were conducted. The cells were incubated on ice for 30 min with 0.5 μg of conjugated antibodies per 1×10\hat{ }5 cells in 100 μl FACS medium. The antibody used: For the antibody corresponding isotype control was used. After two washing steps the stained cells were strained through a mesh and the fluorescence was measured by the Cytoflex Flow Cytometer and analyzed by FACSexpress Version 6.

Activation assay: To assess the activation capacity of the YTS cells with the stimulus of CD48 positive (721.221, BCBL1) and negative (K562) target cells, YTS cells were incubated for 48 h at ratios of 1:1 and 0.5:1 with 50,000 effector cells at 37° C. The cell-free supernatant was used for IFN-γ specific sandwich ELISA. Nunc MaxiSorp™ flat-bottom ELISA plates (Invitrogen) were coated with 1 μg/ml purified anti-IFN-γ (BLG-502402) or anti-TNF-α (BLG-502802) in 50 μl PBS×1 and incubated for 2 h at 37° C. followed by blocking with 200 μl 1% BSA in PBS×1 incubated for 2 h at room temperature (RT). Washing buffer of PBS×1+0.05% Tween-20, was used for washing the wells 3 times. 100 μl of supernatant were incubated within the coated wells at 4° C. overnight. The biotinylated IFN-γ detection antibody (BLG-502504) or TNF-α detection antibody (BLG-502904) was then added at 1 μg/ml in 100 μl 1% BSA in PBS×1 and incubated for 1 h at RT. Finally, streptavidin HRP (016-030-084, Jackson immuno research) 1 μg/ml in 100 μl PBS×1+0.05% Tween-20+1% BSA was incubated for 30 min at RT, and quantification was performed with TMB one component substrate (Southern Biotech).

Killing assay: For assessment of functional killing of seYTS cells radioactive killing assays were employed. For the assay K562, BJAB, and 721.221 cells were labelled with [35S]-Methionine 12 h prior to the assay as target cells. The labelled targets, 5,000 cells/well, were incubated with the various YTS cells at various E:T ratios (1:1, 2:1, 3.75:1, 8:1, 15:1) as effector cells. The assays were performed in 96-U shaped plates at 37° C. for 5 h. After incubation, the plates were centrifuged (1600 rpm, 5 min, 4° C.) and 50 μl of the supernatants were collected and transferred to opaque Opti-plates (Packard). Following the addition of 150 μl scintillation liquid (Perkin Elmer) the plates were analyzed by a micro beta, β-counter (Perkin Elmer). The maximal [35S] release was determined by adding 100 μl of 0.1N NaOH to each target cell line. Spontaneous release of radioactivity was determined in wells containing target cells only. The final specific lysis was calculated as follows: ((radioactive reading—spontaneous release)/(maximal release—spontaneous release))*100=specific lysis.

MTT assay: To assess the proliferation of the irradiated YTS cells, M5655 Sigma thiazolyl blue tetrazolium bromide (MTT) was used. 50 mg MTT was solubilized in 10 ml PBS×1 and sterile filtered. For each condition 25,000 cells per well were seeded at day 0 in quadruplicates in the respective media. After 24 h intervals the cells were incubated with 10 μl of MTT for 3 h in 37° C. up until 72 h after initial seeding. Following the incubation, cells were resuspended in 100 μl DMSO to lyse the stained cells. After cell lysis, the colorimetric changes were measured at 590 nm.

Irradiation: Immune cells were irradiated at 1,000, 2,000, 3,000 and 6,000 cGy.

Mice: All experiments were performed using 6-8 weeks severe combined immunodeficiency disease (SCID)-beige female mice. All mice were housed under Specific pathogen-free (SPF) conditions, in normal light/dark cycles at 22° C.±2° C. All experiments were performed in accordance with the guidelines of the ethics committee of the Hebrew University Medical School (Ein-Kerem, Jerusalem). Every group of mice contained three females (n=3). Xenografts were generated by administering the indicated cells subcutaneously into the left flank region. The mice were monitored daily and sacrificed at any indication of illness such as bristled fur, difficult breathing, or tremor, among others. When tumors reached a maximal volume of 1500 mm3, all mice were sacrificed. No differences were observed between the various groups in their general health at baseline.

### Example 1: Overexpression of NeST in YTS Cell Line

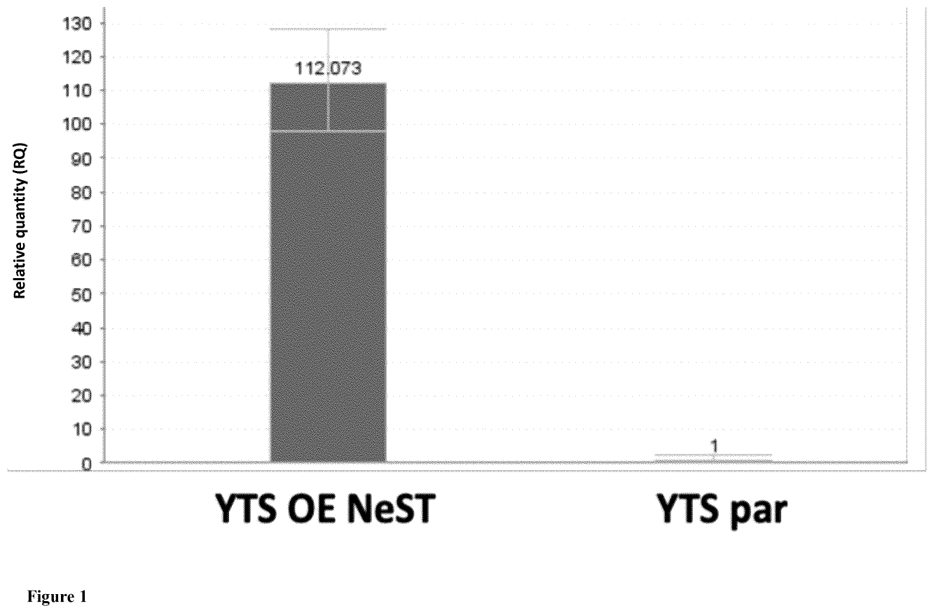

The inventors first stably expressed the IncRNA NeST in the YTS cells via transduction with a lentiviral vector dsRed. As can be seen in FIG. 1, there was a highly elevated expression of NeST in the RT-PCR analysis of the YTS over expressing NeST cells (YTS OE NeST), as compared to parental YTS cells (YTS par).

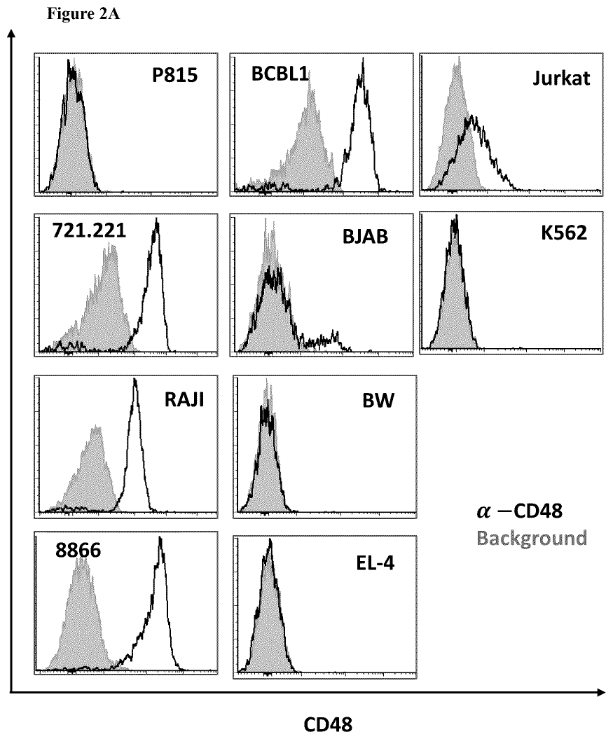

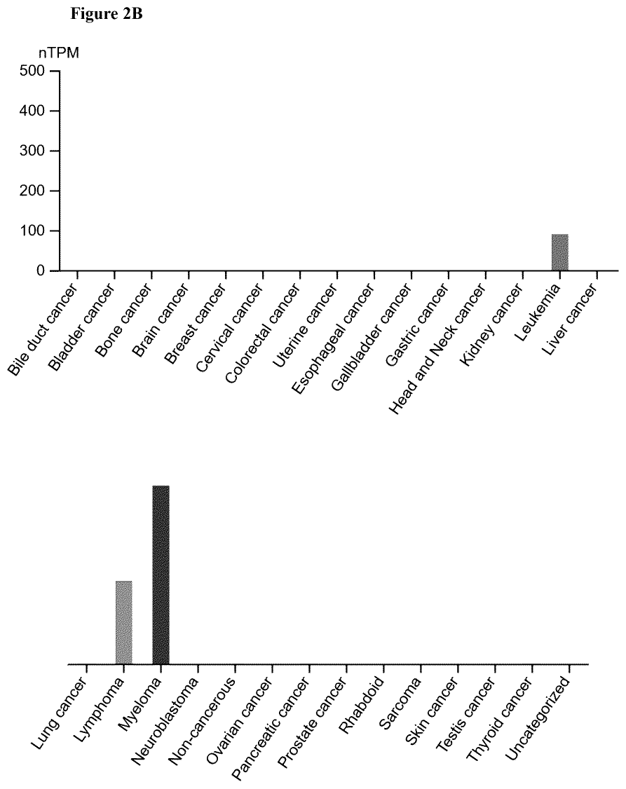

YTS cells are known to kill tumor cells via the interaction between their expressed co-stimulating receptor 2B4 with its ligand present on tumor cells, CD48. Therefore, various cells lines were next screened for expression of CD48. As demonstrated in FIG. 2A, the cell lines; 721.221 (human HLA negative B-Lymphoblastoid cell line), RAJI (human Burkitt lymphoma B cell line), 8866 (human chronic myelogenous leukemia cell line), BCBL-1 (human B cell Non-Hodgkin lymphoma cell line), BJAB (human EBV-negative Burkitt-like B lymphoma cell line), and Jurkat (human T cell leukemia cell line) were all found to express CD48, whereas P815 (murine mastocytoma cell line), BW (murine thymoma cell line BW5147), EL-4 (murine T lymphoma cell line), and K562 (human chronic myelogenous leukemia cell line), were negative for CD48 expression. This correlates with data in the human protein atlas (proteinatlas.org/ENSG00000117091-CD48/cell+line) which shows CD48 highly expressed in lymphoma and myeloma cells, moderately expressed in leukemia cells and essentially absent from all other cancer cell types (FIG. 2B)

### Example 2: Characterization of Cytotoxic Capacity of YTS Cells Overexpressing Nest

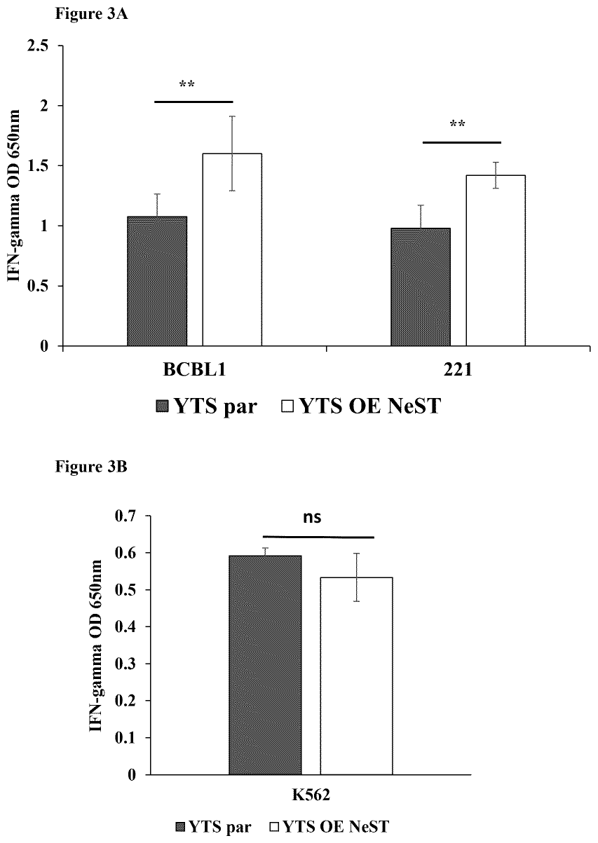

Next, the inventors examined the effect on IFN-γ secretion of activation of YTS NeST cells by coculture with the CD48-expressing cells. YTS NeST cells, or the control YTS parental cells, were incubated with either 721.221 cells, or BCBL-1 cells, at an effector-to-target (E:T) ratio of 0.5:1 for an incubation period of 48 h. IFN-γ levels in the supernatants were then measured by ELISA. There was a significant elevation in IFN-γ production in YTS NeST cells, incubated with both CD48-positive cell lines (FIG. 3A), and no induction in IFN-γ production when YTS NeST cells were incubated with the CD48 non-expressing cell line K562 (FIG. 3B). An increase in IFNG secretion of 84% (0.5:1) and 58% (1:1) was observed in the BCBL1 coculture, which an increase of 55% (0.5:1) and 37% (1:1) was observed in the 721.221 culture.

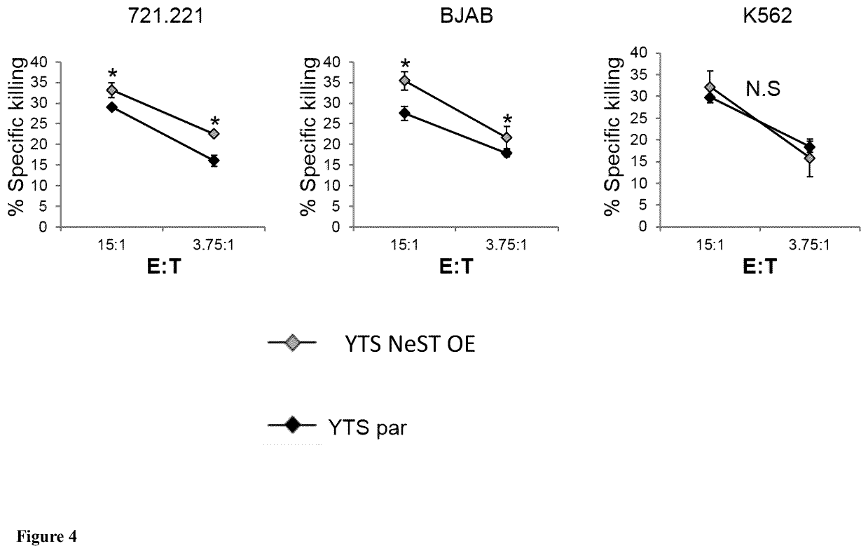

The capacity of YTS NeST cells to kill CD48-positive cell lines was next examined, by using a standard cytotoxic assay, in which the target cells are labeled with a radioactive material (35S methionine) and the number of dead target cells is correlated to the level of released 35S methionine into the medium. As can be seen in FIG. 4, YTS NeST cells producing greater specific killing of 721.221 and BJAB cells than the parental YTS cells. An increase in killing was not observed for the CD48 negative K562 cells. Thus, YTS NeST cells exhibit an increased cytotoxic activity against CD48 expressing tumor cells, which is at least partially, mediated via IFN-γ secretion.

### Example 3: Irradiation Efficacy Studies

As YTS cells are cancerous, the cells must be rendered non-proliferative before they can safely be administered to patients. Clinical trial NCT02944162 examines the transplant of cells of the NK-92 tumor cell line. In this trial, the cells were pre-irradiated with 1,000 cGy of ionizing radiation. The NK-92 cells are CAR-NK cells, and this irradiation renders the cells non-proliferative, preventing the generation of iatrogenic malignancy, but still functional. The inventors irradiated the YTS NeST cells with 1,000, 2,000, 3,000, and 6,000 cGy to assess their functionality and viability after irradiation. The proliferation capacity of the irradiated cells was examined by MTT assay. As can be seen in FIG. 5, YTS NeST cells irradiated with 1000 cGy were still proliferative though at a reduced rate as compared to unirradiated cells. Irradiation dose of 2000 cGy and above, however, rendered the cells non-proliferative. These results indicate that in contrast to the NK-92 cell line, a dose of 1000 cGy is not sufficient to render YTS NeST cells non-cancerous.

The activation induced by CAR constructs is well studied and understood, however, the mechanism by which NeST induces increased IFN-γ secretion and cytotoxicity is not. As such, it was not known if non-proliferative, and in particular irradiated, NK cells would still have enhanced cytotoxicity as compared to the parental cells. To evaluate this, irradiated parental and YTS NeST cells were incubated with BCBL1 cells or 721.221 cells and IFN-γ secretion was measured. YTS NeST cells irradiated with 2000 cGY (FIG. 6A-6B), 3000 cGY (FIG. 6C) and 6000cGy (data not shown) still produced enhanced IFNG secretion when exposed to CD48 expressing cancer cells. The optimal irradiation dose range was found to be between 2000 cGy to 3000 cGy. Surprisingly, coculture of irradiated YTS NeST cells with BCBL1 cells at an E:T ratio of 0.5:1 produced an 84% increased IFN-γ secretion as compared to the irradiated parental cells (FIG. 6A). This is highly unexpected as coculture of the non-irradiated cells with BCBL1 cells at this E:T ratio produced only a 48% increase (FIG. 3A). Thus, irradiation actually produces an improvement in IFN-γ secretion when comparing to the control parental cells. No increase in IFN-γ secretion was observed when the irradiated YTS NeST cells were incubated with the CD48 negative cell line K562 (FIG. 6D), indicating that irradiation does not impair the specificity of the cells.

The killing capacity of the irradiated YTS parental and YTS OE NeST was further examined. As can be seen in FIG. 7A, similar to the non-irradiated cells, the irradiated YTS NeST cells killed both 721.221 and BJAB significantly better than the YTS parental cells. The increased cytotoxic activity of irradiated YTS NeST cells was observed against CD48 expressing cell lines 721.221 and BJAB, and not against the CD48 negative target K562 (FIG. 7B).

### Example 4: In-Vivo Studies Using Irradiated YTS NeST Cells

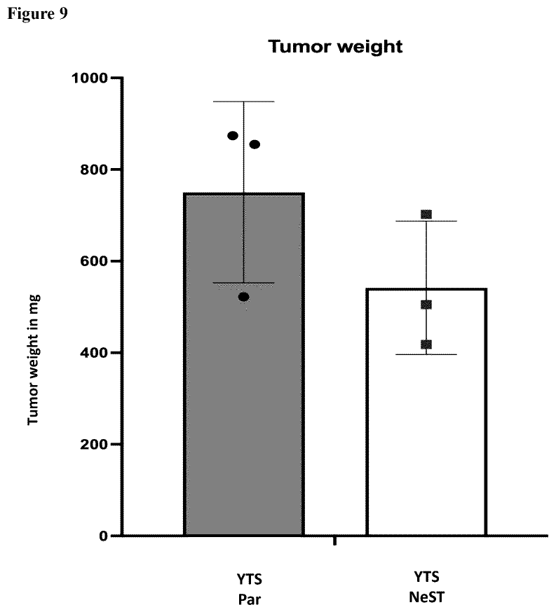

The next objective was to examine the in-vivo effect of irradiated YTS NeST (also named seYTS) cells. 5×10\hat{ }6 YTS cells/mouse (irradiated and not irradiated) with or without NeST overexpression were subcutaneously injected into SCID Beige mice and the health of the mice was observed for 30 days. Unirradiated NK-92 cells were used as a cancerous control. By day 22 all mice administered unirradiated cells (NK-92, YTS parental, seYTS) were dead (FIG. 8). In contrast, all animals administered irradiated cells were not only alive but showed no change in their overall well-being (FIG. 8). Interestingly, the average tumor weight produced by the non-irradiated seYTS cells was lower than that produced by the non-irradiated YTS parental cells, suggesting that the expression of NeST renders the cells less carcinogenic (FIG. 9).

Having established that the irradiated YTS cells were safe, their in vivo efficacy in treating cancer was tested. A murine tumor model was generated by xenografting B-lymphoblastoid cell line 721.221 cells into SCID-Beige mice. The tumor was induced by subcutaneous injection of 10×10\hat{ }6 721.221 cells, when tumors became measurable (day 27), PBS (vehicle), irradiated YTS parental cells (YTSpar), irradiated YTS NeST (seYTS) and irradiated NK-92 cells were intravenously injected to mice bearing the established tumors. 2×10\hat{ }6 cells were irradiated at a dose of 2000 cGy and then suspended in 100p PBS for injection. As shown in FIG. 10, the mice injected with irradiated seYTS exhibited the smallest tumors with the most successful tumor growth repression. NK-92 cells had no effect and were comparable to the vehicle control. Surprisingly, parental YTS cells produced a negative effect, enhancing the tumors growth as compared to vehicle control. In summary, YTS NeST cells, irradiated with 2000 cGY, have an improved anticancer therapeutic potential, compared to both irradiated YTS parental cells and NK-92 cells and furthermore they do not pose a risk of initiating cancer themselves.

Irradiated and non-irradiated NK-92 cells are also made to exogenously express NeST and are tested for increased secretion of IFN-γ in the present of CD48 positive cancer cells and for specific killing of CD48 cancer cells. NeST expression in NK-92 cells also increases specific IFN-γ secretion and specific killing. Irradiated NK-92 NeST cells are also tested in a mouse model of cancer and are able to shrink tumor size as compared to irradiated NK-92 parental cells.

### Example 5: CD16 Expression in YTS Cells

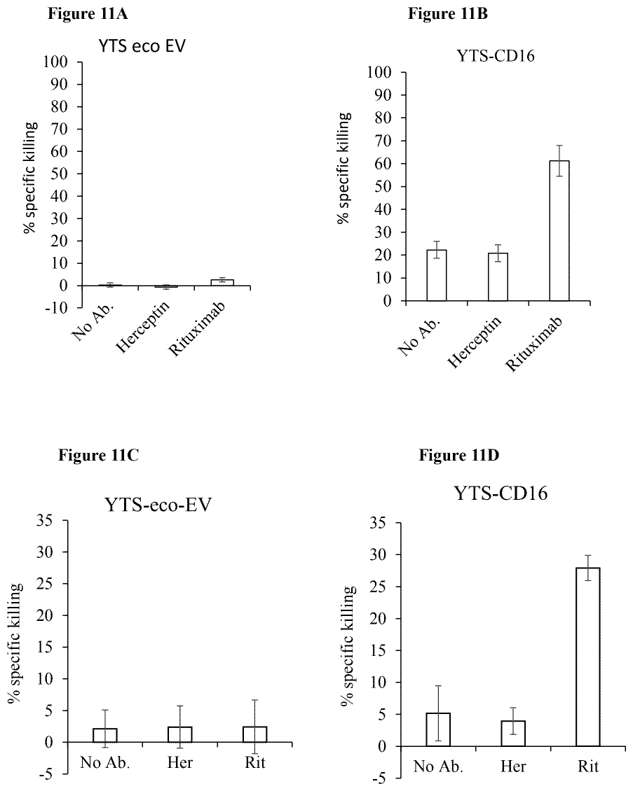

Next, the addition of other anticancer molecules to YTS cells was tested. CD16 is an Fc gamma specific receptor that can induce ADCC in NK cells upon engaging a cell with an antibody engaged to its surface. Raji lymphocytes cells, which are positive for CD20 and negative for Her2, were contacted with Rituximab (anti-CD20) and Herceptin (anti-Her2) antibodies. The cells were then cultured with either control YTS cells (expressing an empty vector) or with YTS cells transfected with full-length CD16. A naturally occurring allelic variant of CD16 comprising a F157V mutation (also known as F158V in the literature) which increases affinity of CD16 for the Fc was used. Specific killing of the Raji cells was monitored. After 3 hours the control YTS cells had produced essentially no specific killing (less than 5% of Raji cells) (FIG. 11A). The CD16 expressing cells in contrast produced a background killing of about 20% of cells (both untreated Raji cells and Herceptin treated cells) and killed over 60% of Rituximab treated cells (FIG. 11B). This demonstrates the high efficacy of CD16 expressing YTS cells.

Primary CLL cells were also tested as the Raji cells had been. An E:T ratio of 20:1 was used, and as before less than 5% specific killing was observed when control YTS cells were used (FIG. 11C). A lower background killing was observed for these cells when YTS-CD16 cells were used; less than 5% specific killing was observed for CLL cells incubated with no antibody or incubated with Herceptin. When Rituximab was used the specific killing with YTS-CD16 cells was almost 30% (FIG. 11D).

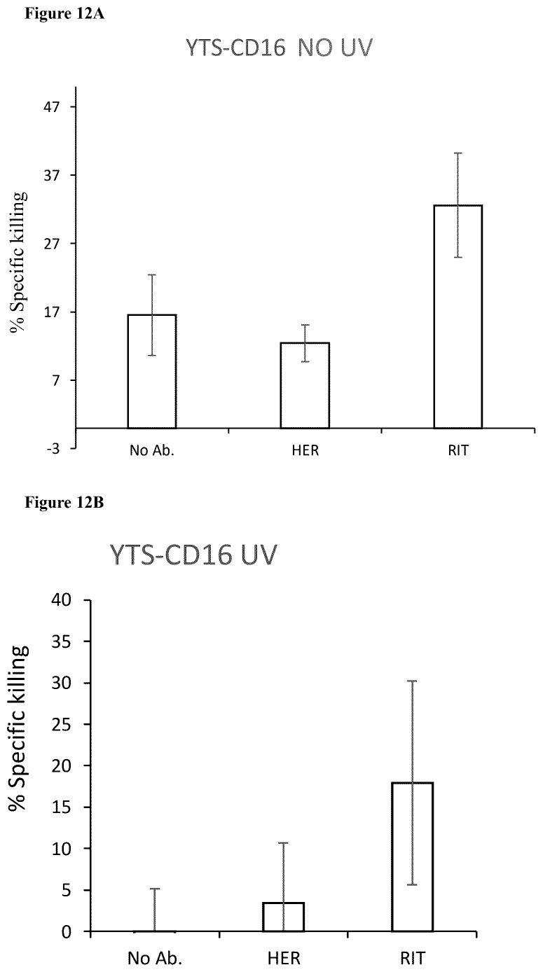

YTS cells expressing CD16 were irradiated at 1000, 2000, 3000 and 6000 cGy and it was confirmed that 2000-3000 cGy is the optimal irradiation to halt cellular division as was observed with the YTS-NeST cells. The ability of 2000 cGy irradiated YTS-CD16 cells to specifically kill CD48 positive cell lines was confirmed, and this irradiation was able to reduce background non-specific killing induced by the YTS-CD16 cells. Raji cells were again used and YTS-CD16 non-irradiated or irradiated cells were cocultured with Raji cells that had been preincubated with no antibody, Herceptin or Rituximab. As before an increased killing was observed for the Rituximab treated cells, but a background non-specific killing was also observed in the control cells (FIG. 12A). When the irradiated YTS-CD16 cells were used specific killing of the Rituximab contact cells was still observed, but the non-specific killing was brought below 5% (FIG. 12B). Thus, irradiation not only renders the YTS cells non-tumorigenic, but it also reduces off-target killing effects.

Additional CD16 constructs are also tested for their ability to induce specific killing when expressed in YTS cells. Constructs bearing mutations to the CD16 ECD which increase Fc binding or reduce enzymatic cleavage are tested. Further, chimeric constructs in which the CD16 ECD and transmembrane domain are linked to immune cell signaling domains (e.g., 2B4, DAP10, CD3Z and FCER1G) are tested. These constructs are also irradiated and specific killing by irradiated cells is confirmed.

A murine tumor model with CD48 positive cancer cells is generated. When tumors become measurable, PBS (vehicle), irradiated YTS parental cells (YTSpar), and irradiated YTS-CD16 cells (WT CD16, mutantECDs and chimeric proteins) are intravenously injected to mice bearing the established tumors. 2×10\hat{ }6 cells are irradiated at a dose of 2000-3000 cGy and are suspended in 100 μl PBS for injection. The mice injected with irradiated YTS-CD16 exhibit the smallest tumors with the most successful growth repression. YTS-CD16 cells are also found to be surprisingly superior to NK-92-CD16 cells both before and after irradiation.

### Example 6: CAR-CD19 Expression in YTS Cells

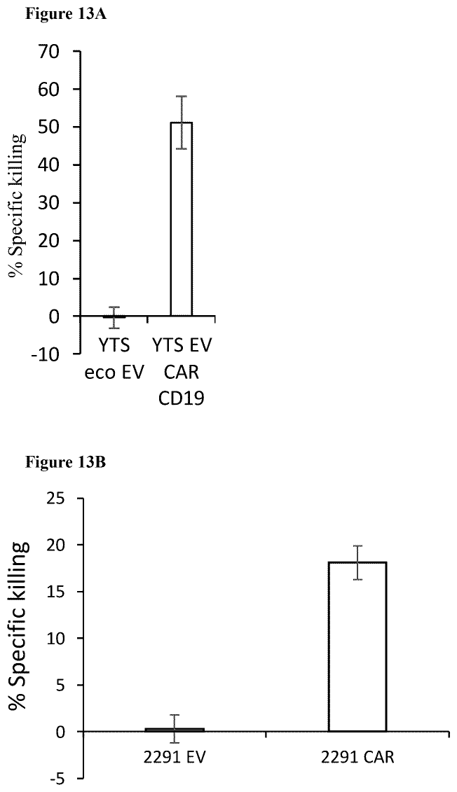

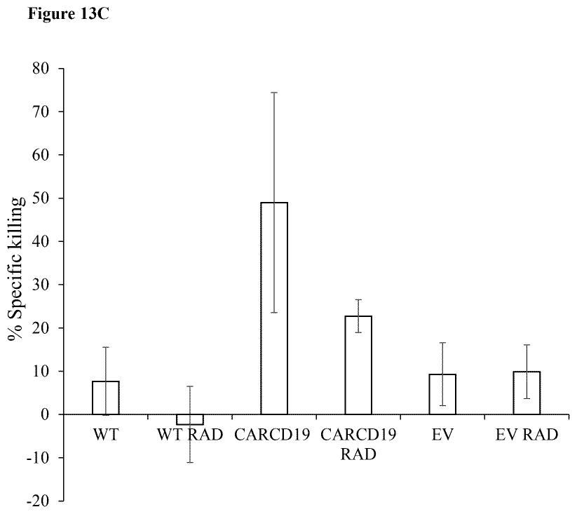

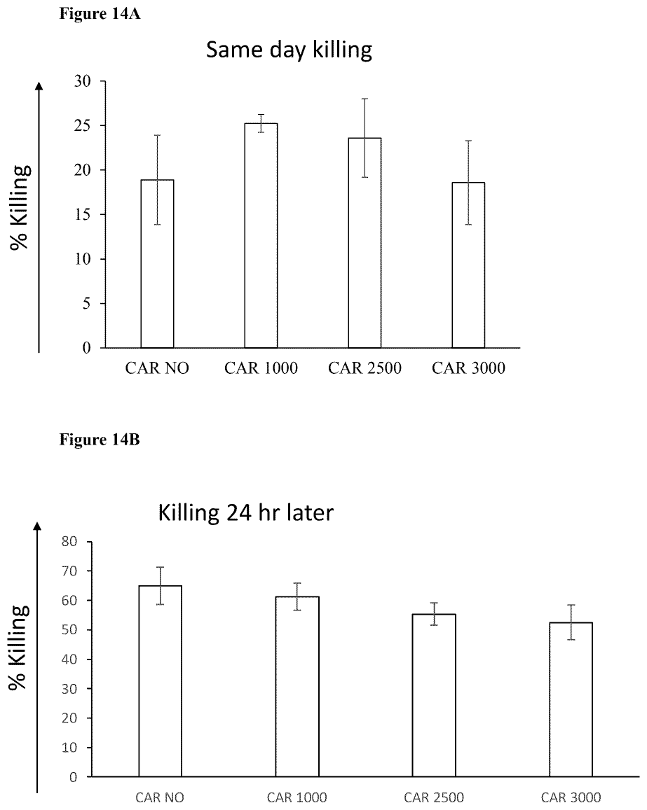

CD19 is a B cell specific antigen that is expressed in the earliest stages of B cell development until plasma cell differentiation. CARs against CD19 are well known and widely used against B cell cancers. Raji lymphocytes cells are CD19 positive. These cells were cultured with either control YTS cells (expressing an empty vector) or with YTS cells transfected with an anti-CD19 CAR (FMC63-28-3z, containing the FMC63 scFV against CD19, the CD28 costimulatory domain and the CD3zeta signaling domain, Addgene #135991). Specific killing of the Raji cells was monitored. After 3 the control YTS cells had produced essentially no specific killing but the CAR-CD19 expressing cells in contrast produced over 50% killing of target cells (FIG. 13A). This demonstrates the high efficacy of CAR-CD19 expressing YTS cells. Primary CLL cells were also tested and YTS-CAR-CD19 cells again produced robust specific killing (FIG. 13B). This increased killing was also observed when the YTS CAR-CD19 cells were first irradiated (FIG. 13C)

To further characterize the ideal level for irradiating YTS CAR-CD19 cells, cells were irradiated with either 1000, 2500 or 3000 cGy. Non-irradiated cells were used as control. The cells were then cultured with Raji cells and killing was measured at two time points: the same day (2-3 hours after mixing) and 24 hours later. On the same day, non-irradiated YTS CAR-CD19 cells produced ~18% specific killing, surprisingly cells irradiated with 1000 or 2500 cGY produced superior killing on the same day (~25% and ~23%, respectively) (FIG. 14A). 24 hours after coculture 55% or greater killing was observed for all cells (FIG. 14B).

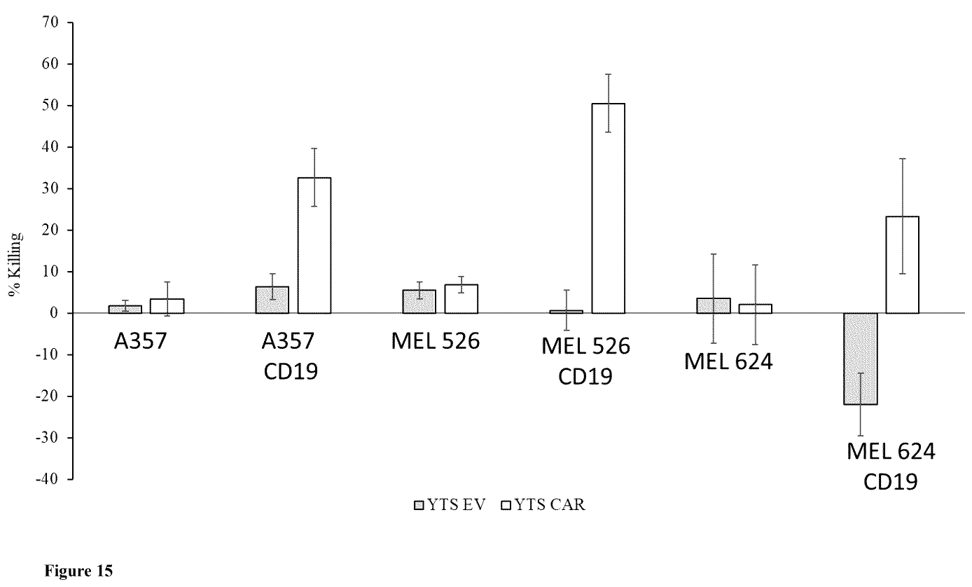

YTS CAR-CD19 cells were effective not only in killing hematopoietic cancers but could also be employed to kill solid tumors. Three melanoma cell lines which are negative for CD48 and CD19 were selected to test the efficacy of the CAR expressing YTS cells. Coculture of the YTS cells with unmodified melanoma cells produced very low levels of background killing (less than 10%) regardless of whether the YTS cells express the anti-CD19 CAR or whether they were made to express empty vector (EV) (FIG. 15). However, when the melanoma cells were made to overexpress CD19, a significant increase in specific killing was observed in the coculture with the CAR expressing YTS cells. This provides a proof of concept that the YTS CAR-CD19 cells can be used against solid tumors which are not the natural targets of this cell lines so long as the tumors are CD19 positive.

A murine tumor model with CD48 and CD19 positive cancer cells is generated. When tumors become measurable, PBS (vehicle), irradiated YTS parental cells (YTSpar), and irradiated YTS-CAR-CD19 cells are intravenously injected to mice bearing the established tumors. 2×10\hat{ }6 cells are irradiated at a dose of 2000-3000 cGy and are suspended in 100 μl PBS for injection. The mice injected with irradiated YTS-CAR-CD19 exhibit the smallest tumors with the most successful growth repression. YTS-CAR-CD19 cells are also found to be surprisingly superior to NK-92-CAR-CD19 cells both before and after irradiation.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A pharmaceutical composition comprising a cell of the YTS immortalized NK cell line exogenously expressing 1) an anti-CD19 chimeric antigen receptor (CAR), 2) CD16 or a functional fragment thereof capable of binding an Fc or 3) the lncRNA NeST.

2. (canceled)

3. The pharmaceutical composition of claim 1, wherein said CD16 is CD16a and comprises the amino acid sequence of SEQ ID NO: 2.

4. The pharmaceutical composition of claim 1, wherein said CD16 is CD16a comprising SEQ ID NO: 2 with a F157V mutation, an S197P mutation or both.

5. The pharmaceutical composition of claim 1, wherein said cell comprises a chimeric protein comprising an extracellular domain of CD16, a transmembrane domain and an intracellular immune cell signaling domain.

6. The pharmaceutical composition of claim 1, wherein said cell is a non-proliferating cell.

7. A pharmaceutical composition comprising a non-proliferating cell of an immortalized natural killer (NK) cell line exogenously expressing the lncRNA NeST.

8. The pharmaceutical composition of claim 7, wherein at least one of:
a. said cell comprises a transmembrane receptor for CD48;
b. said cell comprises the transmembrane receptor 2B4; and
c. said cell comprises a lentiviral expression vector encoding said NeST.

9. (canceled)

10. The pharmaceutical composition of claim 7, wherein said NK cell line is YTS.

11. The pharmaceutical composition of claim 7, wherein said NeST comprises the nucleotide sequence as set forth in SEQ ID NO: 1.

12. (canceled)

13. The pharmaceutical composition of claim 6, wherein said non-proliferating cell is an irradiated cell obtainable by a method comprising:
a. providing a cell of the YTS immortalized NK cell line exogenously expressing CD16 or a functional fragment thereof capable of binding an Fc or an anti-CD19 CAR; and
b. irradiating said cell of step (a) with an ionizing radiation dose between 1500 cGY to 6000 cGY, optionally between 2000 cGY to 3000 cGY.

14. The pharmaceutical composition of claim 7, wherein said non-proliferating cell is obtainable by a method comprising:
(a) providing a cell of an immortalized NK cell line exogenously expressing a long non-coding RNA (lncRNA) NeST; and,
(b) irradiating said cell of step (a) with an ionizing radiation dose between 1500 cGY to 6000 cGY, optionally between 2000 cGY to 3000 cGY;
thereby, producing a non-proliferating cell.

15. (canceled)

16. (canceled)

17. (canceled)

18. (canceled)

19. A method for treating a CD48 expressing cancer or an autoimmune disease in a subject in need thereof, the method comprising administering to said subject a pharmaceutical composition of claim 1, thereby treating a CD48 expressing cancer or an autoimmune disease.

20. The method of claim 19, wherein at least one of:
a. said CD48 expressing cancer is selected from: hematopoietic cancer, hepatocellular carcinoma, glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, and melanoma;
b. said CD48 expressing cancer is a hematopoietic cancer;
c. said CD48 expressing cancer is lymphoma; and
d. said CD48 expressing cancer is a CD19 expressing cancer.

21. (canceled)

22. (canceled)

23. (canceled)

24. (canceled)

25. The pharmaceutical composition of claim 19, wherein said method is a method of treating an autoimmune disease selected from systemic sclerosis, lupus, dermatomyositis, multiple sclerosis (MS) and rheumatoid arthritis (RA).

26. (canceled)

27. (canceled)

28. (canceled)

29. A method for producing a non-proliferating natural killer (NK) cell, the method comprising:
I. (a) providing a cell of an immortalized NK cell line exogenously expressing a long non-coding RNA (lncRNA) NeST; and,
(b) irradiating said cell of step (a) with ionizing radiation; or
II. (a) providing a cell of the immortalized NK cell line YTS exogenously expressing CD16 or a functional fragment thereof capable of binding an Fc or an anti-CD19 CAR; and,
(b) irradiating said cell of step (a) with ionizing radiation;
thereby, producing a non-proliferating NK cell.

30. The method of claim 29, wherein said cell of an immortalized NK cell line exogenously expressing a long non-coding RNA (lncRNA) NeST:
a. comprises a transmembrane receptor for CD48;
b. comprises the transmembrane receptor 2B4;
c. is the cell line YTS;
d. comprises the nucleotide sequence as set forth in SEQ ID NO: 1; or
e. comprises a lentiviral expression vector encoding said NeST.

31. (canceled)

32. (canceled)

33. (canceled)

34. (canceled)

35. (canceled)

36. The method of claim 29, wherein
a. said CD16 is CD16a and comprises the amino acid sequence of SEQ ID NO: 2;
b. said CD16 is CD16a comprising SEQ ID NO: 2 with an F157V mutation, an S197P mutation or both; or
c. said cell comprises a chimeric protein comprising an extracellular domain of CD16, a transmembrane domain and an intracellular immune cell signaling domain.

37. (canceled)

38. (canceled)

39. (canceled)

40. The method of claim 29, wherein said irradiating is with an ionizing radiation dose between 1500 cGY to 6000 cGY, optionally between 1500 cGY to 3500 cGY.

41. (canceled)

42. (canceled)

43. The method of claim 29, wherein said non-proliferating cell is further characterized by: increased cytotoxic activity against a target cell, increased inflammatory activity, or both, as compared to a control cell of said immortalized NK cell not exogenously expressing NeST or a control YTS cell not expressing CD16 or a functional fragment thereof capable of binding an Fc or an anti-CD19 CAR, optionally wherein said target cell is a cancer cell expressing CD48, CD19 or both.

44. (canceled)

45. The method of claim 29, further comprising after said irradiating measuring proliferation of said cell and selecting a cell that does not proliferate.

46. (canceled)

47. (canceled)


SEQUENCE LISTING (29 sequences)

SEQ ID NO: 1 - DNA, 1979 bp, Homo sapiens
  source 1..1979: mol_type = genomic DNA
ctgcaatttcaggtagcttttctgactcttaaagagatctcaagtataccttcagagaaatgccagcaaaaactgtagtcatttgggaaggaataagcctggaagaaaaagatacaacgaactagcacaacgaggagtttgaaaagttcatgacagctcacagctgatgatggtggcaatcttaaggatacagaaagctcattcctcatgcagggaagaagaaaatattctaaagaagagataagcatattccatgaaatcaaaaaagcataaaacgctggaggagaagtcagtagcaagcagctaagacaacatggtacatgtggctagaagcccaccaactgctaacaaccagctggagggtaagtcaaaagctatgagagcccagaaggagaacaattatttagacctggaggggccagttgggaaagtctttgaagggtcttcagaaaatggaccaaaagaaaagaggaaaagtcattctactgagaggtgtttgtgtaatcaaagcaaccgaggcctaagaactcacaaaagattagaaaagggtaaaggatgcctggaaagccggactaggaaaaggctgtgaagggccctgaaggtcacatgtcacatgttgcctgcagcatgcagaatcaaaacatgataaatcatgtcttgaatcatgacatatgaagcatgacacacgaatcatgacatgtgacatgttgaatgtcacatgtcacatgccgtaggcagccatcaagagtatttgaaaggaaagtgtataatcaggcctgtgctttagtaagacaatttgtatagtgttcagtgtggattaaaatggagaacacaggtatcagtttgacatcgacagtacaggaaaccatttgggtttcttgtacaccaaaatattaaaacatacacgcaaacacaccaacacattaaaatcttgagagagagggccaaaagttctttgtcgctggactaaaagtggaaataaatgtttacaacctctaaggtggattaagctaagaatttggagttgttggcaaaactgccacataaaatttcagacatgcaaagaaatgcaatcgtagacttatgcacatacttccaccagagaagggaatctgggcagagtgttcaaagcttaagaaattcctttgctcttcaccaagaagagccaacagagttttcgttgaatcaactcttatattcccttaaaaagaagttgatagtgttgtggaaaactcacccattgaagtgacaacaaaaacagcaacccagtatgagttaactggtattatgttaaagggacaagtaactgcacttttgacacacatcctctaaaaggtagggtgggagtgtttcaaagaaccaagtctgagtaatagaaaagcatataccactttcccatatacacgttcgagcatgggggagcaggaagctgggtaattgaatgcagttgtgcacactggcaattttcccgccacataccatttcttcggtgtctctcccaaaattctcctcctaagtggcacatccctttattgatcatctttccccatactgatcatcctcagcaatcatgtgagttattagtctaagtatttgtttttctcaaatatggtcttttaactactccctaaatattttatatatacatagtttgttttcccaatttaactacaagcatttaaaaaatgggaatatttgtctatatttctgtaatttttcatcatgccttatatggtatataaagatcaacagcaataaaacaacaacagtaataatagctaagaccttttgaatactaactgtatgttcagcattatttcaacagctttacaggtattaactgatttaattattacaacaacggcatgaagtagatactactgttgtcatcagtttatacttgaggaaactgagttatgggatggttaattatcttttccatggtcacagagccattaaatgtatgatctcagacagtc

SEQ ID NO: 2 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 3 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 4 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 5 - protein, 24 aa, Homo sapiens
LCARPRRSPAQEDGKVYINMPGRG

SEQ ID NO: 6 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 42 aa, Homo sapiens
RLKIQVRKAAITSYEKSDGVYTGLSTRNQETYETLKHEKPPQ

SEQ ID NO: 8 - protein, 192 aa, Homo sapiens
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 9 - protein, 21 aa, Homo sapiens
VSFCLVMVLLFAVDTGLYFSV

SEQ ID NO: 10 - protein, 208 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 11 - protein, 208 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQ

SEQ ID NO: 12 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDVYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 13 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDVHIPKATLKDSGSYFCRGLVGSKNVSSETVNITITQGLAVSTISSFSPPGYQVSFCLVMVLLFAVDTGLYFSVKTNI

SEQ ID NO: 14 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVPTISSFFPPGYQVSFCLVMVLLFAVDTGLYFSVKTNIRSSTRDWKDHKFKWRKDPQDK

SEQ ID NO: 15 - protein, 233 aa, synthetic construct
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVPTISSFSPPGYQVSFCLVMVLLFAVDTGLYFSVKTNI

SEQ ID NO: 16 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDVYIPKATLKDSGSYFCRGLFGSKNVSSETVNITITQGLAVPTISSFFPPGYQVSFCLVMVLLFAVDTGLYFSVKTNIRSSTRDWKDHKFKWRKDPQDK

SEQ ID NO: 17 - protein, 233 aa, synthetic construct
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDVHIPKATLKDSGSYFCRGLVGSKNVSSETVNITITQGLAVPTISSFSPPGYQVSFCLVMVLLFAVDTGLYFSVKTNI

SEQ ID NO: 18 - protein, 192 aa, Homo sapiens
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDVYIPKATLKDSGSYFCRGLFGSKNVSSETVNITITQGLAVSTISSFFPPGYQ

SEQ ID NO: 19 - protein, 208 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDVYIPKATLKDSGSYFCRGLFGSKNVSSETVNITITQGLAVSTISSFFPPGYQ

SEQ ID NO: 20 - protein, 192 aa, synthetic construct
GMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDVHIPKATLKDSGSYFCRGLVGSKNVSSETVNITITQGLAVSTISSFSPPGYQ

SEQ ID NO: 21 - protein, 208 aa, synthetic construct
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDVHIPKATLKDSGSYFCRGLVGSKNVSSETVNITITQGLAVSTISSFSPPGYQ

SEQ ID NO: 22 - protein, 192 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVPTISSFFPPGYQ

SEQ ID NO: 23 - protein, 208 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVPTISSFFPPGYQ

SEQ ID NO: 24 - protein, 192 aa, synthetic construct
GMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVPTISSFSPPGYQ

SEQ ID NO: 25 - protein, 208 aa, synthetic construct
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVPTISSFSPPGYQ

SEQ ID NO: 26 - protein, 192 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDVYIPKATLKDSGSYFCRGLFGSKNVSSETVNITITQGLAVPTISSFFPPGYQ

SEQ ID NO: 27 - protein, 208 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDVYIPKATLKDSGSYFCRGLFGSKNVSSETVNITITQGLAVPTISSFFPPGYQ

SEQ ID NO: 28 - protein, 192 aa, synthetic construct
GMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDVHIPKATLKDSGSYFCRGLVGSKNVSSETVNITITQGLAVPTISSFSPPGYQ

SEQ ID NO: 29 - protein, 208 aa, synthetic construct
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDVHIPKATLKDSGSYFCRGLVGSKNVSSETVNITITQGLAVPTISSFSPPGYQ